(19) United States
(12) Patent Application Publication
(10) Pub. No.: US 2010/0069683 A1
(43) Pub. Date: Mar. 18, 2010

(54) Organic metal compound and process for preparing optically-active alcohols using the same

(75) Inventors: Takashi Miki, Saitama (JP); Junichi Hori, Saitama (JP); Toshihide Takemoto, Saitama (JP); Noriyuki Utsumi, Saitama (JP); Takeaki Katayama, Saitama (JP); Masahito Watanabe, Saitama (JP); Kunihiko Murata, Saitama (JP)

Correspondence Address: EDWARDS ANGELL PALMER & DODGE LLP, P.O. BOX 55874, BOSTON, MA, 02205, US

(73) Assignee: Kanto Kagaku Kabushiki Kaisha, Chuo-Ku (JP)

(21) Appl. No.: 12/583,943

(22) Filed: Aug. 28, 2009

Related U.S. Application Data
(62) Division of application No. 12/218,874, filed on Jul. 18, 2008, now abandoned.

(30) Foreign Application Priority Data
Jul. 19, 2007 (JP) 2007-188339

(51) Int. Cl. C07C 37/00

(52) U.S. Cl. 568/799

(57) ABSTRACT

The present invention provides an asymmetric reduction catalyst effective in preparing optically-active alcohol compounds having various functional groups, and a process for preparing optically-active alcohol compounds using said asymmetric reduction catalyst.

The organic metal compound of the present invention is represented by the following general formula (1):

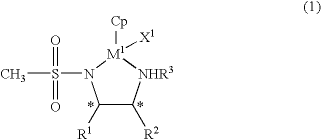

wherein R¹ and R² may be mutually identical or different, and are an alkyl group, a phenyl group, a naphthyl group, a cycloalkyl group, or an alicyclic ring formed by binding R¹ and R², which may have a substituent; R³ is a hydrogen atom or an alkyl group; Cp is a cyclopentadienyl group, which may have a substituent, bound to M¹ via a π bond; X¹ is a halogen atom or a hydrido group; M¹ is rhodium or iridium; and * denotes asymmetric carbon.

This application is a divisional of U.S. Ser. No. 12/218,874, filed on Jul. 18, 2008.

### TECHNICAL FIELD

The present invention relates to a novel organic metal compound and a process for preparing optically-active alcohols using the same.

### BACKGROUND ART

To date, various preparation processes of optically-active alcohols using metal complexes as catalysts have been reported. In particular, processes in which optically-active alcohols are synthesized from ketone compounds by reductive process using ruthenium complexes as catalysts under the presence of base are being actively investigated. These processes are classified into “asymmetric hydrogenation” wherein hydrogen is used as a hydrogen source, and “asymmetric reduction” wherein organic substances and metal hydrides are used as a hydrogen source; their characteristics are as follows.

With respect to asymmetric hydrogenation wherein optically-active alcohols are obtained from ketones by asymmetric hydrogenation using hydrogen as a reducing agent, and to catalysts used therein, for example, JP No. 2731377 reports a process for preparing an optically-active alcohol by hydrogenation of a ketone compound under the presence of base, using a complex in which BINAP (2,2′-bis(diphenylphosphino)-1,1′-binaphthyl) and DMF are coordinated to ruthenium as well as diphenylethylenediamine as catalysts. While this catalyst had extremely high activity, there were problems regarding the applicability of the ketone substrates, namely, that the hydrogenation reaction did not progress efficiently or the enantiomeric excess was insufficient depending on the structure of the ketone compound.

Therefore, to expand the range of applicable ketone substrates, catalysts with different structures were developed. In concrete terms, the following reactions are reported: using a ruthenium catalyst having TsDPEN (N-toluenesulfonyl-1,2-diphenylethylenediamine) as a ligand, the reaction of 4-chromanone (J. Am. Chem. Soc. Vol. 128, p. 8724 (2006)) and the reaction of α-chloroketones (Org. Lett. Vol. 9, p. 255 (2007)); asymmetric hydrogenation of α-hydroxyketone using an iridium catalyst having MsDPEN (N-methanesulfonyl-1,2-diphenylethylenediamine) as a ligand (WO 2006/137195, Org. Lett. Vol. 9, p. 2565 (2007)). With these catalyst systems, there is no need to add bases so that the type of ketone substrates that can be used for the reaction has been expanded. However, there still remain ketone substrates with which hydrogenation is difficult. In addition, these catalyst systems are easily affected by slight amounts of impurities existing in ketone substrates, which is problematic when actual industrial application is considered.

In contrast to the above, asymmetric reduction that uses an organic substance as a hydrogen source does not require a pressure-resistant container, so that there is no limitation in the production equipment and the process is advantageous in terms of cost; thus, a number of reports have been published. In particular, in the case of asymmetric ruthenium catalysts that have a diamine ligand having a sulfonyl amide group as an anchor (JP No. 2962668), it was reported that a wide range of ketones can be asymmetrically reduced. There are also several reports on rhodium catalysts or iridium catalysts that have a diamine ligand having a sulfonyl amide group as an anchor (J. Org. Chem. Vol. 64, p. 2186 (1999), Chem. Lett. p. 1199 (1998), Chem. Lett. p. 1201 (1998), JP A No. 11-335385, WO 98/42643, WO 00/18708). These rhodium and iridium catalysts exhibit characteristic catalytic performances; when formic acid is used as a hydrogen source, these catalysts are reported to demonstrate efficacy in asymmetric reduction of imines (WO 00/56332) and α-haloketone (WO 2002/051781).

However, catalytic efficiencies of these catalytic reactions are not sufficient in many cases, and formic acid used as a hydrogen source has a corrosive nature. In addition, upon execution of the reaction, formic acid must be used after neutralization with an organic base such as triethylamine; however, in the process of mixing formic acid with triethylamine, significant heat is generated and this heat of neutralization must be removed, which leads to a significant problem in quantity synthesis. Moreover, the type of ketones that can be applied is limited.

Furthermore, there is a report on asymmetric reduction of aromatic ketones such as acetophenones, indanone, and acetonaphthone using an asymmetric ruthenium catalyst and sodium formate as a hydrogen source (Org. Biomol. Chem. Vol. 2, p. 1818 (2004)); however, no investigation has been made on the preparation of optically-active alcohols from aromatic ketones having a functional group.

With respect to asymmetric reduction of ketones using formate as a hydrogen source, for example, there is a report on asymmetric reduction of aromatic ketones using an iridium catalyst having TsCYDN (N-tosyl-1,2-cyclohexanediamine) as a ligand (Chem. Commun. p. 4447 (2005)). However, the S/C ratio (molar ratio of substrate/catalyst) which is an index for catalytic activity is at the highest 1000, and there is no investigation on optically-active alcohols having industrially-effective functional groups.

The use of CsDPEN, which is a DPEN ligand having a camphorsulfonyl group, has also been reported (Synlett p. 1155 (2006)), showing that iridium catalysts have fairly good catalytic activity compared to ruthenium or rhodium catalysts; however, their S/C ratio is at the highest 1000, and examples of their application to ketone substrates with a functional group are limited to acetophenones and propiophenones having a functional group on an aryl group, acetylbenzofurane, and trans-chalcone. In addition, the use of a rhodium complex having CsDPEN as a ligand has been reported (WO 2004/110976); however, only acetonaphthone is disclosed as a specific example of ketones. As a camphor which constitutes CsDPEN, an optically active form must be used; however, camphorsulfonyl chloride necessary for the synthesis of CsDPEN is expensive, and its (R)-(−)-form is especially expensive. The fact that as a ligand, an asymmetric ligand other than diamine is required significantly increases the cost of the catalysts, which leads to an increase in the cost of optically-active alcohols obtained from the catalytic reactions.

Thus, although the synthesis of optically-active alcohols having a functional group is industrially very important, the processes thus far reported, which use ruthenium complexes as catalysts, have a problem of insufficient catalytic activity and they need to use a formic acid/triethylamine mixture solution, of which the handling is very difficult. While the asymmetric reduction using iridium complexes as catalysts solves these problems, it has problems in that the catalysts are expensive and there is a limitation in the structure of ketone substrates having applicable functional groups. Namely, in the majority of structures of applicable ketone substrates, the position at which a functional group binds is an aromatic group; in the structures wherein a functional group is present at side chains such as the α-position, β-position and γ-position of the aromatic ketone, efficient reduction has not yet been achieved.

### DISCLOSURE OF THE INVENTION

### Problem to be Solved by the Invention

Therefore, the object of the present invention is to provide a novel organic metal compound used as an asymmetric reduction catalyst applicable to the preparation of optically-active alcohols having various industrially-effective functional groups, with high efficiency, low cost and in an easy-to-handle manner, which can solve the above-mentioned problems of conventional technologies in obtaining optically-active alcohols using ketones as raw materials, and to provide a process for preparing optically-active alcohol compounds using such asymmetric catalyst.

### Means of Solving the Problem

In order to solve the above-mentioned problems, the present inventors have found that, during their devoted research, a novel organic metal compound having iridium or rhodium and a N-methanesulfonyl-1,2-diamine ligand exhibits a catalytic reaction to enable highly-enantioselective and highly-efficient asymmetric reduction of a wide range of ketones, and the inventors have accomplished this invention after further advance in the research.

Namely, the present invention relates to an organic metal compound represented by the general formula (1):

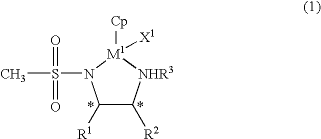

wherein R¹ and R² may be mutually identical or different, and are an alkyl group, a phenyl group, a naphthyl group, a cycloalkyl group, or an alicyclic ring formed by binding R¹ and R², which may have a substituent; R³ is a hydrogen atom or an alkyl group; Cp is a cyclopentadienyl group bound, which may have a substituent, to M¹ via a π bond; X¹ is a halogen atom or a hydrido group; M¹ is rhodium or iridium; and * denotes asymmetric carbon.

The present invention also relates to said organic metal compound, wherein R³ is a hydrogen atom and M¹ is iridium in the general formula (1).

Furthermore, the present invention relates to said organic metal compound, wherein X¹ is a halogen atom in the general formula (1).

The present invention also relates to a process for preparing optically active alcohols by asymmetric reduction of ketone substrates, wherein a ketone substrate is reacted with a hydrogen-donating compound under the presence of an organic metal compound represented by the general formula (2):

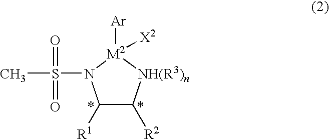

wherein R¹ and R² may be mutually identical or different, and are an alkyl group, a phenyl group, a naphthyl group, a cycloalkyl group, or an alicyclic ring formed by binding R¹ and R², which may have a substituent; R³ is a hydrogen atom or an alkyl group; Ar is a cyclopentadienyl group or a benzene ring group, which may have a substituent, bound to M² via a it bond; X² is a hydrido group or an anionic group; M² is rhodium or iridium; n is 0 or 1, and X² is absent when n is 0;
and * denotes asymmetric carbon.

Furthermore, the present invention relates to said process, wherein R³ is a hydrogen atom and M² is iridium in the general formula (2).

The present invention also relates to said process, wherein a formate is used as a hydrogen-donating compound, and water or water/organic solvent is used as a solvent.

Furthermore, the present invention relates to said process, wherein a phase-transfer catalyst is additionally added.

The present invention also relates to said process, wherein a ketone having a hydroxyl group at the α-position or the β-position of the ketone is asymmetrically reduced.

Furthermore, the present invention relates to said process, wherein a ketone having a halogen at the α-position or the β-position of the ketone is asymmetrically reduced.

The present invention also relates to said process, wherein a ketone having a carbon-carbon multiple bond at the α-position or the β-position of the ketone is asymmetrically reduced.

Furthermore, the present invention relates to said process, wherein a ketone having an ester group at the α-position or the β-position of the ketone, or a ketone having an ester group at the carbonyl carbon of the ketone is asymmetrically reduced.

The present invention also relates to said process, wherein a ketone having a carboxylic amide group at the α-position or the β-position of the ketone, or a ketone having a carboxylic amide group at the carbonyl carbon of the ketone is asymmetrically reduced.

Furthermore, the present invention relates to said process, wherein a ketone having an amino group at the α-position or the β-position of the ketone is asymmetrically reduced.

The present invention also relates to said process, wherein 1,2-diketone or 1,3-diketone is asymmetrically reduced.

Furthermore, the present invention relates to said process, wherein a cyclic ketone is asymmetrically reduced.

### EFFECTS OF THE INVENTION

When the organic metal compound of the present invention is used as a catalyst, reaction of many ketone substrates proceeds with high efficiency, and optically-active alcohols having a high purity can be obtained. In addition, in many of catalytic asymmetric reactions, slight amounts of impurities present in a ketone substrate tend to affect results of the catalytic reaction; however, according to the process of the present invention, the reaction is not disturbed without purification of commercially-available ketone substrates, and an optically-active alcohol of interest can be obtained in high yield. Moreover, when the inventive catalyst is used in a two-phase reaction system using a hydrogen-donating compound as the hydrogen source in a solvent such as formate (water, water/organic solvent and the like), ketones which conventionally have not been well reacted can be reduced with high efficiency and high selectivity, to provide optically-active alcohols. Namely, it is now possible to efficiently obtain optically-active alcohols from ketones having a substituent at the n-position, such as β-hydroxypropiophenone and β-chloropropiophenone, or ketones having a heterocyclic ring, such as ethyl 3-oxo-3-(4-pyridyl)propionate, ethyl 3-oxo-3-(2-thienyl) propionate, and 3-hydroxy-1-(2-thienyl)-propanone, the reaction of which was conventionally very slow even when hydrogen or formic acid was used as the hydrogen source and an asymmetric ruthenium, rhodium or iridium catalyst having a MsDPEN ligand of similar structure was used. Since the structure of the catalyst used in the present invention is simple and its synthesis cost is low, industrial reduction of ketones can be performed with low cost.

According to the present invention, only by mixing a hydrogen-donating compound (formic acid, formate and the like), a certain organic metal compound (iridium complex or rhodium complex), and a ketone substrate into a solvent (water, water/organic solvent and the like), the asymmetric reduction of the ketone proceeds rapidly, enabling highly-enantioselective and highly-efficient asymmetric reduction of ketones having functional groups of which highly efficient asymmetric reduction was impossible with conventional catalysts, so that various optically-active alcohols can be easily obtained with simple operation and low cost.

### BEST EMBODIMENT OF THE INVENTION

The organic metal compound of the present invention is represented by the above general formula (1), and the organic metal compound used in the process of the present invention is represented by the above general formula (2). R¹ and R² in the general formulae (1) and (2) are an alkyl group, a phenyl group, a naphthyl group, or a cycloalkyl group, which may have a substituent, and R¹ and R² may be mutually identical or different.

Examples of the alkyl group which may have a substituent include, for example, an alkyl group with a carbon number from 1 to 10 such as methyl group, ethyl group, n-propyl group, isopropyl group, n-butyl group, sec-butyl group, and tert-butyl group; examples of the phenyl group which may have a substituent include an phenyl group having an alkyl group with a carbon number from 1 to 5 such as phenyl group, 4-methylphenyl group, and 3,5-dimethylphenyl group, a phenyl group having a halogen atom such as 4-fluorophenyl group and 4-chlorophenyl group, and a phenyl group having an alkoxy group such as 4-methoxyphenyl group. In addition, examples of the naphthyl group which may have a substituent include naphthyl group, 5,6,7,8-tetrahydro-1-naphthyl group, and 5,6,7,8-tetrahydro-2-naphthyl group; examples of the cycloalkyl group which may have a substituent include cyclopentyl group, and cyclohexyl group. Furthermore, R¹ and R² may be an unsubstituted or substituted alicyclic ring formed by binding R¹ and R². Examples of such alicyclic ring include cyclopentane ring and cyclohexane ring. Among them, it is particularly preferable that R¹ and R² are both phenyl group, or a cyclohexane ring formed by binding R¹ and R².

Concrete examples of R³ in the general formulae (1) and (2) include an alkyl group with a carbon number from 1 to 5 such as methyl group and ethyl group as well as a hydrogen atom; hydrogen atom is particularly preferred.

Concrete examples of Cp in the general formula (1) include cyclopentadienyl group, methylcyclopentadienyl group, 1,2-dimethylcyclopentadienyl group, 1,3-dimethylcyclopentadienyl group, 1,2,3-trimethylcyclopentadienyl group, 1,2,4-trimethylcyclopentadienyl group, 1,2,3,4-tetramethylcyclopentadienyl group and 1,2,3,4,5-pentamethylcyclopentadienyl group, 1,2,3,4-tetramethyl-5-ethylcyclopentadienyl group, 1,2,3,4-tetramethyl-5-isopropylcyclopentadienyl group, 1,2,3,4-tetramethyl-5-n-propylcyclopentadienyl group, 1,2,3,4-tetramethyl-5-n-butylcyclopentadienyl group, 1,2,3,4-tetramethyl-5-sec-butylcyclopentadienyl group, 1,2,3,4-tetramethyl-5-tert-butylcyclopentadienyl group, 1,2,3,4-tetramethyl-5-phenylcyclopentadienyl group, 1,2,3,4-tetramethyl-5-trifluoromethylcyclopentadienyl group, 1,2,3,4-tetramethyl-5-pentafluoroethyl-cyclopentadienyl group, and 1,2,3,4-tetramethyl-5-pentafluorophenyl-cyclopentadienyl group.

Concrete examples of Ar in the general formula (2) include cyclopentadienyl group, methylcyclopentadienyl group, 1,2-dimethylcyclopentadienyl group, 1,3-dimethylcyclopentadienyl group, 1,2,3-trimethylcyclopentadienyl group, 1,2,4-trimethylcyclopentadienyl group, 1,2,3,4-tetramethylcyclopentadienyl group and 1,2,3,4,5-pentamethylcyclopentadienyl group, 1,2,3,4-tetramethyl-5-ethylcyclopentadienyl group, 1,2,3,4-tetramethyl-5-isopropylcyclopentadienyl group, 1,2,3,4-tetramethyl-5-n-propylcyclopentadienyl group, 1,2,3,4-tetramethyl-5-n-butylcyclopentadienyl group, 1,2,3,4-tetramethyl-5-sec-butylcyclopentadienyl group, 1,2,3,4-tetramethyl-5-tert-butylcyclopentadienyl group, 1,2,3,4-tetramethyl-5-phenylcyclopentadienyl group, 1,2,3,4-tetramethyl-5-trifluoromethylcyclopentadienyl group, 1,2,3,4-tetramethyl-5-pentafluoroethyl-cyclopentadienyl group, 1,2,3,4-tetramethyl-5-pentafluorophenyl-cyclopentadienyl group, as well as unsubstituted benzene, and a benzene having an alkyl group such as toluene, o-, m- or p-xylene, o-, m- or p-cymene, 1,2,3-, 1,2,4- or 1,3,5-trimethylbenzene, 1,2,4,5-tetramethylbenzene, 1,2,3,4-tetramethylbenzene, pentamethylbenzene, hexamethylbenzene, and the like.

X¹ in the general formula (1) is a halogen atom or a hydrido group; examples of the halogen atom include fluorine atom, chlorine atom, bromine atom or iodine atom. X² in the general formula (2) is a hydrido group or an anionic group, and the anionic group in this specification includes halogen atoms. In addition, in the general formula (2), n is 0 or 1, and X² is absent when n is 0.

Concrete examples of X² in the general formula (2) include hydrido group, cross-linked oxo group, fluorine atom, chlorine atom, bromine atom, iodine atom, tetrafluoroborate group, tetrahydroborate group, tetrakis[3,5-bis(trifluoromethyl)phenyl]borate group, acetoxy group, benzoyloxy group, (2,6-dihydroxybenzoyl)oxy group, (2,5-dihydroxybenzoyl)oxy group, (3-aminobenzoyl)oxy group, (2,6-methoxybenzoyl)oxy group, (2,4,6-triisopropylbenzoyl)oxy group, 1-naphthalenecarboxylate group, 2-naphthalenecarboxylate group, trifluoroacetoxy group, trifluoromethanesulfonimide group, nitromethyl group, nitroethyl group, methanesulfonyl group, ethanesulfonyl group, n-propanesulfonyl group, isopropanesulfonyl group, n-butanesulfonyl group, fluoromethanesulfonyl group, difluoromethanesulfonyl group, trifluoromethanesulfonyl group, pentafluoroethanesulfonyl group, and hydroxyl group. Among them, trifluoromethanesulfonyl group, hydrido group, fluorine atom, chlorine atom, bromine atom or iodine atom are particularly preferred.

Each of M¹ in the general formula (1) and M² in the general formula (2) is either iridium or rhodium, and is preferably iridium. It can be said that an organic metal compound represented by the general formula (1) or (2) has a structure wherein an ethylenediamine compound (CH₃SO₂NHCHR¹CHR²NHR³) which is a bidentate ligand is bound to a metal. Examples of the ethylenediamine compound which constitutes the organic metal compound represented by the general formula (1) or (2) include, for example, N-methanesulfonyl-1,2-diphenylethylenediamine (MsDPEN), N-methanesulfonyl-1,2-cyclohexanediamine (MsCYDN), N-methyl-N′-methanesulfonyl-1,2-diphenylethylenediamine, and N-methyl-N′-methanesulfonyl-1,2-cyclohexanediamine. Among them, MsDPEN and MsCYDN are particularly preferred.

As a process for preparing the organic metal compounds represented by the general formulae (1) and (2), those described in J. Org. Chem. Vol. 64, p. 2186 (1999) or Chem. Lett. p. 1201 (1999) can be used. In concrete terms, the compounds can be synthesized by the reaction of pentamethylcyclopentadienyl rhodium complex or pentamethylcyclopentadienyl iridium complex with N-methanesulfonyl-1,2-diamine ligand.

The process for preparing optically active alcohols of the present invention is performed by reacting a ketone compound with a hydrogen-donating compound, under the presence of an iridium catalyst or a rhodium catalyst which is an organic metal compound represented by the general formula (2). The reaction is performed by, for example, mixing and stirring an iridium or rhodium catalyst of the general formula (2), a ketone compound, water and a formate. In cases when the mixture of a ketone substrate with a catalyst must be accelerated, for example when the ketone substrate is a solid, an organic solvent may be added. The amount of the catalyst used in this case is, in terms of molar ratio of the ketone compound to the iridium or rhodium catalyst, i.e., S/C(S denotes substrate and C denotes catalyst), preferably between 50 and 10,000 from the viewpoint of practical application, but not limited thereto.

As a reaction solvent, water or organic solvents may be used; water alone, or water with an organic solvent is preferred. Examples of the organic solvent include, alcoholic solvents such as methanol, ethanol, 2-propanol, 2-methyl-2-propanol and 2-methyl-2-butanol, ether solvents such as tetrahydrofuran (THF), diethylether, tert-butyl methyl ether (TBME) and cyclopentyl methyl ether (CPME), heteroatom-containing solvents such as DMSO, DMF and acetonitrile, aromatic hydrocarbon solvents such as benzene, toluene and xylene, aliphatic hydrocarbon solvents such as pentane, hexane and cyclohexane, halogen-containing hydrocarbon solvents such as methylene chloride, and ester solvents such as ethyl acetate; these solvents may be used alone, or 2 or more kinds of the solvents may be used in combination. Furthermore, a mixed solvent of the above solvents with other solvents may also be used.

A hydrogen-donating compound (hydrogen source) is a compound which can donate hydrogen to ketones in the present invention, including, for example, formic acid, formate, formic acid ester, alcohol (methanol, ethanol, propanol, isopropanol, butanol, benzylalcohol and the like), and hydroquinone. A hydrogen-donating compound is preferably formic acid, formate or formic acid ester, and is more preferably formate from the viewpoints of operability, reaction yield and optical purity.

As a formate, a salt of formic acid with an alkaline metal or alkaline earth metal, etc. may be used. Preferable concrete examples of the formate include lithium formate, sodium formate, potassium formate, cesium formate, magnesium formate, and calcium formate. The formate is particularly preferably sodium formate or potassium formate. Regarding the amount of the formate used, when expressed by a molar ratio, at least the equimolar amount relative to the ketone substrate is necessary. Considering the practical applicability, the range from 1 to 10 molar equivalents is preferred. The concentration of the formate is selected optimally considering the balance between the amount of the ketone substrate reacted and the size of the reaction equipment. The higher the concentration of the formate, the higher the reaction rate.

If necessary, a phase-transfer catalyst may be added in the reaction. Examples of the phase-transfer catalyst include tetrabutylammonium fluoride, tetrabutylammonium chloride, tetrabutylammonium bromide, tetrabutylammonium iodide, tetrabutylammonium hydroxide, tetramethylammonium fluoride, tetramethylammonium chloride, tetramethylammonium bromide, tetramethylammonium iodide, tetramethylammonium hydroxide, benzyltrimethylammonium fluoride, benzyltrimethylammonium chloride, benzyltrimethylammonium bromide, benzyltrimethylammonium iodide, benzyltrimethylammonium hydroxide, tetraethylammonium fluoride, tetraethylammonium chloride, tetraethylammonium bromide, tetraethylammonium iodide, tetraethylammonium hydroxide, tetrapropylammonium fluoride, tetrapropylammonium chloride, tetrapropylammonium bromide, tetrapropylammonium iodide, tetrapropylammonium hydroxide, hexadecyltrimethylammonium fluoride, hexadecyltrimethylammonium chloride, hexadecyltrimethylammonium bromide, hexadecyltrimethylammonium iodide, hexadecyltrimethylammonium hydroxide, phenyltrimethylammonium fluoride, phenyltrimethylammonium chloride, phenyltrimethylammonium bromide, phenyltrimethylammonium iodide, phenyltrimethylammonium hydroxide, dodecyltrimethylammonium fluoride, dodecyltrimethylammonium chloride, dodecyltrimethylammonium bromide, dodecyltrimethylammonium iodide, dodecyltrimethylammonium hydroxide, benzyltriethylammonium fluoride, benzyltriethylammonium chloride, benzyltriethylammonium bromide, benzyltriethylammonium iodide, and benzyltriethylammonium hydroxide. The amount of the phase-transfer catalyst added relative to the ketone substrate is preferably in the range from 0.01 to 10 molar equivalents. By the addition of a phase-transfer catalyst, reactivity and enantioselectivity of the ketone substrate can be improved.

The reaction temperature is not particularly limited; considering the economic efficiency, it is preferably in the range from −30 to 60° C., and more preferably in the range from 20 to 60° C. Since the reaction time varies depending on the kind of reaction substrate, concentration, S/C ratio, reaction conditions such as temperature and pressure, and the kind of catalyst, reaction conditions may be set so that the reaction completes within several minutes to several days. In particular, reaction conditions are preferably set so that the reaction completes within 5 to 24 hr. Purification of reaction products can be optionally performed using a known method such as column chromatography, distillation, and re-crystallization.

In the process for preparing optically-active alcohols of the present invention, it is not essential to add acid or base into a reaction system; accordingly, hydrogenation reaction of ketone compounds proceeds rapidly without the addition of acid or base. Needless to say, acid or base may be added; a small amount of acid or base may be optionally added depending on, for example, the structure of reaction substrate and purity of the reagent used.

Both of the chiral carbons at two positions in the organic metal compound represented by the general formula (1) or (2) must be (R) form or (S) form, in order to obtain an optically-active alcohol. By selecting either (R) form or (S) form, an optically-active alcohol with a desired absolute configuration can be obtained with high selectivity.

Preferable concrete examples of the organic metal compound of the invention, or the organic metal compound used in the process of the invention include Cp*IrCl[(S,S)-MsDPEN], Cp*IrCl[(R,R)-MsDPEN], Cp*IrCl[(S,S)-MsCYDN], Cp*IrCl[(R,R)-MsCYDN], Cp*Ir (OTf)[(S,S)-MsDPEN], Cp*Ir(OTf)[(R,R)-MsDPEN], Cp*Ir(OTf)[(S,S)-MsCYDN], Cp*Ir(OTf)[(R,R)-MsCYDN], Cp*RhCl[(S,S)-MsDPEN], Cp*RhCl[(R,R)-MsDPEN], Cp*RhCl[(S,S)-MsCYDN], and Cp*RhCl[(R,R)-MsCYDN]. These organic metal compounds are preferably used as the catalyst for the preparation of optically-active alcohols, together with the above-described hydrogen-donating compounds.

In the process of the present invention, by using an iridium or rhodium catalyst, it is possible to prepare an optically-active alcohol having a halogen atom by the asymmetric reduction of a ketone having a halogen atom at the α- or β-position, or it is possible to prepare an optically-active diol by the asymmetric reduction of a ketone having a hydroxyl group at the α- or β-position. In particular, conventionally it was difficult to efficiently asymmetrically-hydrogenate or reduce ketones having a halogen group and a hydroxyl group at the β-position or ketones having a heterocyclic ring, using a ruthenium catalyst with a diamine ligand. Using the process of the present invention, halogen-substituted optically-active alcohols and optically-active diols can be obtained with high efficiency for the first time, and the efficacy of the present invention is demonstrated by the fact that these alcohols and diols can be easily derivatized into flooxetine or duroxetine that are asymmetric medical agents. Moreover, it is also possible to prepare an optically-active alcohol having an olefin site or acethylene site by hydrogenating a ketone having an olefin site (double bond) or an acethylene site (triple bond) at the α- or β-position, or to prepare an optically-active hydroxyester or hydroxyamide by hydrogenating a ketone having an ester group or a carboxylic amide group at the α- or β-position or at the carbonyl carbon of the ketone. Furthermore, it is possible to prepare an optically-active aminoalcohol by hydrogenating a ketone having an amino group at the α- or β-position, and to prepare optically-active 1,2-diol and 1,3-diol from 1,2-diketone and 1,3-diketone, respectively. Furthermore, an optically-active alcohol having a ring structure can be prepared from a cyclic ketone such as 4-chromanone. Thus, the process of the present invention is extremely useful.

Representative examples of ketone substrates applicable to the process for preparing optically-active alcohols of the present invention are listed below; however, the process of the present invention is not limited to these compounds.

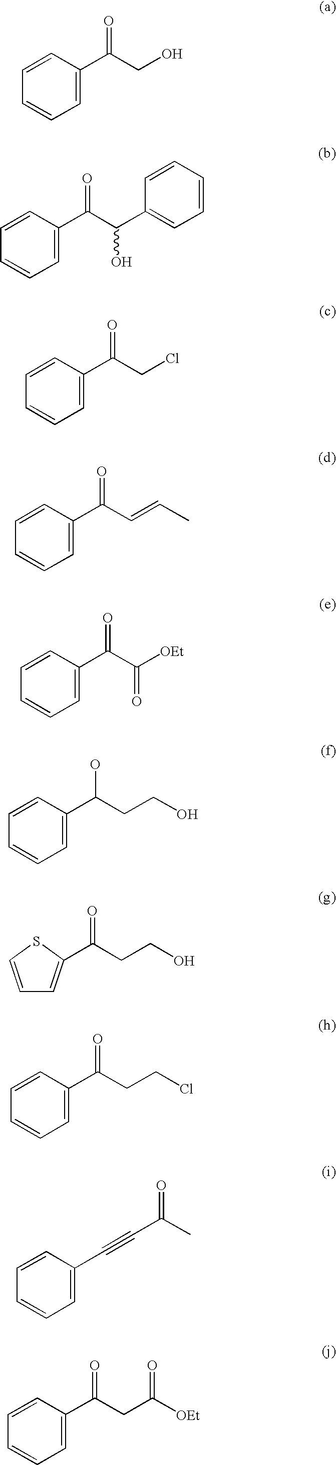

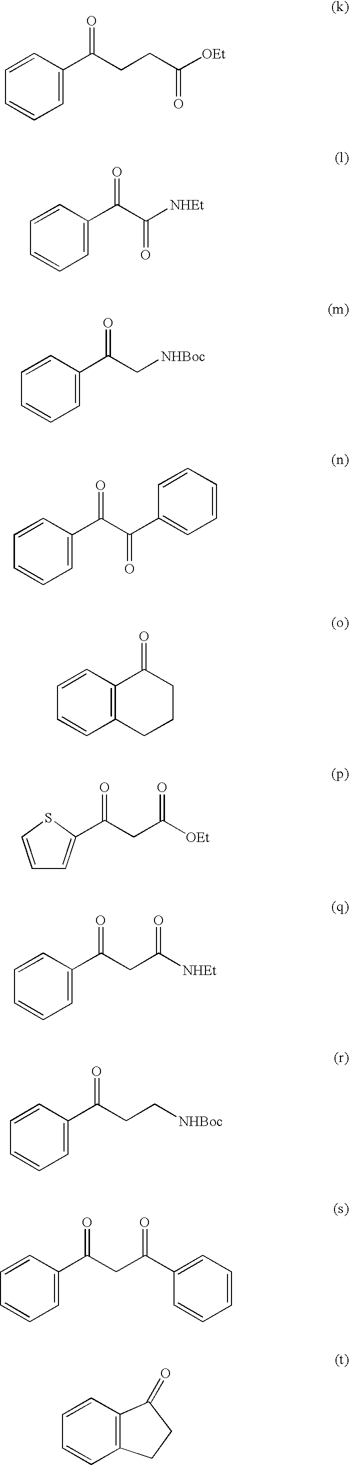

### EXAMPLES

In the following, the present invention is illustrated in more detail by way of examples and comparative Examples, but the present invention is not limited to these examples.

Meanwhile, reactions described in the following examples and comparative Examples were performed under an inert gas atmosphere such as argon gas or nitrogen gas. As the water used in the reactions, those treated by ion-exchange resin were used. Of the ketone substrates listed in Tables 1 to 3, with respect to the following substrates, commercially-available reagents were used as they were: acetophenone, α-hydroxyacetophenone, β-hydroxypropiophenone, α-chloroacetophenone, β-chloropropiophenone, 4-chromanone, ethyl benzoylacetate, ethyl 3-oxo-3-(2-fluorophenyl)propionate, methyl 3-benzoylpropionate, and 1,1,1-trifluoroacetone. Ethyl 3-oxo-3-(4-pyridyl)propionate was synthesized in accordance with the method described in JACS. Vol. 67, p. 1468 (1945), and ethyl 3-oxo-3-(2-thienyl)-propionate was synthesized in accordance with the method described in EP751427 A1. For the identification of complex and reactant, a nuclear magnetic resonator (NMR) was used, wherein the signal of tetramethylsilane (TMS) as the internal standard material was set as δ=0 (δ indicates chemical shift). The conversion ratio from a ketone substrate to an alcohol compound and the enantioselectivity were measured using gas chromatography (GC) or high-performance liquid chromatography (HPLC). As NMR apparatus, JNM-ECX-400P (JEOL Ltd.) was used; as GC apparatus, GC-17A (Shimadzu Corporation) was used. As HPLC apparatus, LC-10ADVP (Shimadzu Corporation) was used.

In the reaction using acetophenone, α-chloroacetophenone, or β-chloropropiophenone as a ketone substrate, CHIRASIL DEX CB (GC Column from CHROMPACK; 0.25 mm×25 m, DF=0.25 μm) was used for the measurement. In the reaction using α-hydroxyacetophenone or β-hydroxypropiophenone as a ketone substrate, CHIRALCEL OB (HPLC column from DAICEL CHEMICAL INDUSTRIES, LTD.; 0.46 cm×25 cm) was used for the measurement. In the reaction using 4-chromanone, CHIRALCEL OJ-H(HPLC column from DAICEL CHEMICAL INDUSTRIES, LTD.; 0.46 cm×25 cm) was used for the measurement. In the reaction using ethyl benzoylacetate or ethyl 3-oxo-3-(2-thienyl)propionate, α-(benzoylamino)acetophenone, and α-(benzyloxycarbonylamino)acetophenone, CHIRALCEL OD (HPLC column from DAICEL CHEMICAL INDUSTRIES, LTD.; 0.46 cm×25 cm) was used for the measurement. In the reaction using ethyl 3-oxo-3-(4-pyridyl)propionate, CHIRALCEL OD-H(HPLC column from DAICEL CHEMICAL INDUSTRIES, LTD.; 0.46 cm×25 cm) was used for the measurement. In the reaction using 2-hydroxy-1-(2-furyl)ethan-1-one, CHIRALPAKAS-H(HPLC column from DAICEL CHEMICAL INDUSTRIES, LTD.; 0.46 cm×25 cm) was used for the measurement.

### Example 1

### Synthesis of Cp*IrCl[(S,S)-MsDPEN]

319 mg (1.10 mmol) of (S,S)-MsDPEN (MW: 290.4) and 398 mg (0.5 mmol) of [Cp*IrCl₂]₂ (MW: 796.6) were introduced in a 50 mL Schlenk tube, and the mixture was subjected to argon substitution. 15 mL of 2-propanol was added and dissolved, then 0.3 mL (2.2 mmol) of triethylamine and 2 mol equivalents of (S,S)-MsDPEN were introduced, and the resulting mixture was stirred at room temperature for 7 hr. After the solvent was distilled off under reduced pressure, 15 mL of methylene chloride was added, and the resulting methylene-chloride solution was transferred to a separating funnel and washed with the addition of 20 mL of water. The aqueous phase was extracted three times with 15 mL of methylene chloride and combined with the organic phase. 5 g of Na₂SO₄ was added and the resulting mixture was stirred for a while, then the supernatant was filtered through a glass filter, and the filtrate was transferred to a 100 mL eggplant-shaped flask. Na₂SO₄ was washed twice with 20 mL of methylene chloride. The methylene chloride was distilled off under reduced pressure to give 645 mg of Cp*IrCl[(S,S)-MsDPEN]. Yield: 990.

¹H NMR (400 Mz, CDCl₃) δ (ppm) 1.78 (s, 15H, C₅(CH₃)₅), 2.41 (s, 3H, CH₃ of Ms), 3.79 (brd, 1H, CHN), 4.11 (brd, 1H NH₂), 4.52 (m, 2H, SO₂NCH, NH₂), 6.96-7.34 (m, 10H, aromatic ring).

The ¹H NMR data indicated that the obtained compound was the title compound.

### Example 2

### Synthesis of Cp*IrCl[(S,S)-MsCYDN]

500 mg (2.60 mmol) of (S,S)-MsCYDN (MW: 192.3) and 1.035 g (1.30 mmol) of [Cp*IrCl₂]₂ (MW: 796.6) were introduced in a 50 mL Schlenk tube, and the mixture was subjected to argon substitution. 25 mL of 2-propanol was added and dissolved, then 0.72 mL (5.2 mmol) of triethylamine was introduced, and the resulting mixture was stirred at room temperature for 0.5 hr. After the solvent was distilled off under reduced pressure, the obtained residue was washed in 20 mL of diisopropylether. The solvent was distilled off under reduced pressure to give 1.88 g (65 wt % content) of Cp*IrCl[(S,S)-MsCYDN] in which 2.9 equivalents of triethylamine (including triethylamine hydrochloride) is coordinated to the complex. Yield: 85%.

¹H NMR (400 Mz, CDCl₃) δ (ppm) 1.2-2.2 (m, 8H, C₆ ring), 1.41 (t, Et₃N), 1.67 (s, 15H, C₅(CH₃)₅), 1.83 (s, 3H, CH₃ of Ms), 2.64 (brd, 1H, NH₂), 2.84 (brd, 1H, NCH), 3.10 (q, Et₃N), 3.4 (m, 1H, NH₂), 3.4 (m, 1H, SO₂NCH) 4.35 (m, 1H, NH₂)

The ¹H NMR data indicated that the obtained compound was the title compound.

### Example 3

### Synthesis of Cp*RhCl[(R,R)-MsDPEN]

470 mg (1.62 mmol) of (R,R)-MsDPEN (MW: 290.4) and 500 mg (0.809 mmol) of [Cp*RhCl₂]₂ (MW: 618.08) were introduced in a 50 mL Schlenk tube, and the mixture was subjected to argon substitution. 15 mL of 2-propanol was added and dissolved, then 0.45 mL (3.2 mmol) of triethylamine was introduced, and the resulting mixture was stirred at room temperature for 7 hr. After the solvent was distilled off under reduced pressure, 15 mL of methylene chloride was added, and the resulting methylene-chloride solution was transferred to a separating funnel and washed with the addition of 20 mL of water. The aqueous phase was extracted three times with 15 mL of methylene chloride and combined with the organic phase. 5 g of Na₂SO₄ was added and the resulting mixture was stirred for a while, and the supernatant was filtered through a glass filter, and the filtrate was transferred to a 100 mL eggplant-shaped flask. Na₂SO₄ was washed twice with 20 mL of methylene chloride. The methylene chloride was distilled off under reduced pressure to give 945 mg of Cp*RhCl[(R,R)-MsDPEN]. Yield: 100%.

¹H NMR (400 Mz, CDCl₃) δ (ppm) 1.80 (s, 15H, C₅(CH₃)₅), 2.41 (s, 3H, CH₃ of Ms), 3.36 (brd, 1H, NH₂), 3.82 (brd, 1H, NCH), 3.97 (brd, 1H, NH2, 4.17 (d, 1H, SO₂NCH), 6.8-7.4 (m, 10H, aromatic ring)

The ¹H NMR data indicated that the obtained compound was the title compound.

### [Reference Example]

Cp*Ir[(S,S)-MsDPEN], Cp*Ir (OTf) [(S,S)-MsDPEN], Cp*IrCl[(S,S)-TsDPEN], Cp*IrCl[(R,R)-TsCYDN], RuCl[(R,R)-TsDPEN] (p-cymene), and RuCl[(R,R)-MsDPEN] (p-cymene) were synthesized in conformity with the methods described in JACS. Vol. 128, p. 8724 (2006) and Org. Lett. ASAP article (Jul. 11, 2007).

### Asymmetric Reduction

Using the catalysts obtained in the above-described examples and reference examples, various ketone substrates were asymmetrically reduced as shown in the formula below. Results are shown in Tables 1 to 3. Figures in Tables 1 to 3 indicate yield of the products, and figures in the parentheses indicate enantiomeric excess (%) of the products. The numbers in Examples and Comparative examples shown below represent the combination of the symbol of substrates (A-P) with the number of catalyst systems (1-15) listed in Tables 1 to 3.

TABLE 1

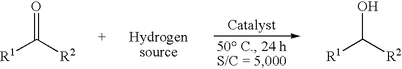

| Substrate | | | | |
|---|---|---|---|---|
| A | B | C | D | E |

| Catalyst system number | Catalyst | Hydrogen source | 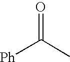 | 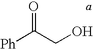 | 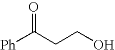 | 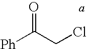 | 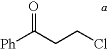 |
|---|---|---|---|---|---|---|---|
| 1. | Cp*IrCl[(S,S)-Msdpen] | HCOOK | 96 (93) | 100 (94) | 99 (93)^{c} | 87 (92) | 94 (85)^{c} |
| 2. | ″ | HCOOH | 56 (75)^{c} | 12 (66) | — | complex | — |
| | | | | | | mixture | |
| 3. | Cp*Ir[(S,S)-Msdpen] | HCOOK | — | 100 (94) | — | — | — |
| 4. | Cp*Ir(0Tf)[(S,S)-Msdpen] | HCOOK | 94 (93) | 97 (90) | — | — | — |
| 5. | ″ | H₂^{g} | — | 100 (96)^{h} | 18 (75)^{d} | 39 (92)^{e} | 12 (77)^{d} |
| | | | | 5 | | | |
| 6. | Cp*IrCl[(R,R)-Mscydn] | HCOOK | 90 (86) | — | 95 (82)^{e} | 92 (82) | — |
| 7. | ″ | HCOOH | 0 | — | — | unknown | — |
| 8. | Cp*RhCl[(S,S)-Msdpen] | HCOOK | 83 (96)^{d} | 100 (98)^{e} | — | 100 (97)^{d} | — |
| 9. | Cp*IrCl[(S,S)-Tsdpen] | HCOOK | 27 (89) | 30 (28) | — | 26 (91) | — |
| 10. | ″ | HCOOH | 33 (60)^{c} | — | — | | — |
| 11. | Cp*IrCl[(R,R)-Tscydn] | HCOOK | — | 10 (68) | — | 29 (80) | — |
| 12. | RuCl[(R,R)-Tsdpen](p-cymene) | HCOOK | — | — | — | 85 (86)^{d} | — |
| 13. | ″ | H₂^{g} | — | — | — | — | 9 (90)^{d} |
| 14. | RuCl[(R,R)-Tsdpen](p-cymene) | HCOOK | — | <1 | 4^{e} | — | — |
| 15. | Cp*IrCl[(R,R)-(R)-Csdpen] | HCOOK | — | 40 (87) | — | — | — |

^{a}Addition of toluene,
^{c}S/C = 500,
^{d}S/C = 1,000,
^{e}S/C = 2,000,
^{g}Asymmetric hydrogenation; in CH₃OH, 60 ° C., 10 atm,
^{h}Purified substrate was used.

TABLE 2

Substrate

| | | | F | G | H |
|---|---|---|---|---|---|
| Catalyst system number | Catalyst | Hydrogen source | 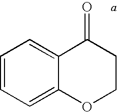 | 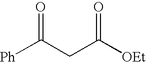 | 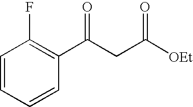 |
| 1. | Cp*IrCl[(S,S)-Msdpen] | HCOOK | 89 (95) | 98 (93) | 100 (59) |
| | | | 99 (97)^{f} | | |
| | | | 96 (98)^{f,l} | | |
| 2. | ″ | HCOOH | 10 (88) | 51 (78)^{d} | — |
| | | | 89 (95)^{e} | | |
| 3. | Cp*Ir[(S,S)-Msdpen] | HCOOK | — | — | — |
| 4. | Cp*Ir(OTf)[(S,S)-Msdpen] | HCOOK | — | — | — |
| 5. | ″ | H₂^{g} | — | 81 (95)^{d} | 40 (65)^{d} |
| 6. | Cp*IrCl[(R,R)-Mscydn] | HCOOK | 44 (94) | — | — |
| 7. | ″ | HCOOH | — | — | — |
| 8. | Cp*RhCl[(S,S)-Msdpen] | HCOOK | — | — | — |
| 9. | Cp*IrCl[(S,S)-Tsdpen] | HCOOK | 19 (94) | — | — |
| 10. | ″ | HCOOH | — | 50 (69)^{d} | — |
| 11. | Cp*IrCl[(R,R)-Tscydn] | HCOOK | 6 (71) | — | — |
| 12. | RuCl[(R,R)-Tsdpen](p-cymene) | HCOOK | — | — | — |
| 13. | ″ | H₂^{g} | | | |
| 14. | RuCl[(R,R)-Msdpen](p-cymene) | HCOOK | 9 (75) | 18 (91) | — |
| 15. | Cp*IrCl[(R,R)-(R)-Csdpen] | HCOOK | — | — | — |

| | Substrate | |
|---|---|---|
| | I | J |
| Catalyst system number | 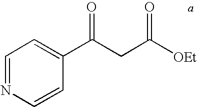 | 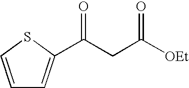 |
| 1. | 100 (84) | — |
| 2. | 11 (80)^{d} | — |
| 3. | — | — |
| 4. | — | 90 (96) |
| 5. | — | 22 (97)^{d} |
| 6. | — | — |
| 7. | — | — |
| 8. | — | — |
| 9. | — | — |
| 10. | — | — |
| 11. | 10 (66)^{d} | — |
| 12. | trace | — |
| 13. | — | — |
| 14. | 16 (75)^{d} | — |
| 15. | — | — |

^{a}Addition of toluene,
^{d}S/C = 1,000,
^{e}S/C = 2,000,
^{f}Addition of TBAB,
^{g}Asymmetric hydrogenation; in CH₃OH, 60° C., 10 atm,
^{l}S/C = 10,000.

TABLE 3

| | | | Substrate | | |
|---|---|---|---|---|---|
| | | | K | L | M |
| Catalyst system number | Catalyst | Hydrogen source | 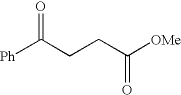 | 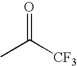 | 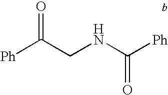 |
| 1. | Cp*IrCl[(S,S)-Msdpen] | HCOOK | — | 73 (87) | 100 (91)^{e} |
| 2. | ″ | HCOOH | — | — | 43^{d,l} |
| 3. | Cp*Ir[(S,S)-Msdpen] | HCOOK | — | — | — |
| 4. | Cp*Ir(OTf)[(S,S)-Msdpen] | HCOOK | 96 (85)^{e} | — | — |
| 5. | ″ | H₂^{g} | 3^{d} | | 55 (85)^{d} |
| 6. | Cp*IrCl[(R,R)-Mscydn] | HCOOK | — | — | — |
| 7. | ″ | HCOOH | — | — | — |
| 8. | Cp*RhCl[(S,S)-Msdpen] | HCOOK | — | — | — |
| 9. | Cp*IrCl[(S,S)-Tsdpen] | HCOOK | — | — | — |
| 10. | ″ | HCOOH | — | — | — |
| 11. | Cp*IrCl[(R,R)-Tscydn] | HCOOK | — | — | — |
| 12. | RuCl[(R,R)-Tsdpen](p-cymene) | HCOOK | — | — | — |
| 13. | ″ | H₂^{g} | — | — | — |
| 14. | RuCl[(R,R)-Tsdpenl(p-cymene) | HCOOK | — | — | — |
| 15. | Cp*IrCl[(R,R)-(R)-Csdpen] | HCOOK | — | — | — |

| | Substrate | | |
|---|---|---|---|
| | N | O | P |
| Catalyst system number | 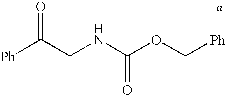 | 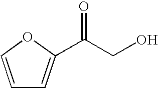 | 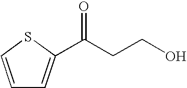 |
| 1. | 100 (96)^{e} | 100 (94)^{d} | 72 (91)^{e} |
| 2. | 9^{d,l} | — | — |
| 3. | — | — | — |
| 4. | — | — | — |
| 5. | 46 (87)^{d} | 12 (70)^{e} | — |
| 6. | — | — | — |
| 7. | — | — | — |
| 8. | — | — | — |
| 9. | — | — | — |
| 10. | — | — | — |
| 11. | — | — | — |
| 12. | — | — | — |
| 13. | — | — | — |
| 14. | — | — | — |
| 15. | — | — | — |

^{a}Addition of toluene,
^{b}Adition of toluene and THF,
^{d}S/C = 1,000,
^{e}S/C = 2,000,
^{f}Addition of TBAB,
^{g}Asymmetric hydrogenation; in CH₃OH, 60° C., 10 atm,
ⁱ30° C.

### Example A-1

### Asymmetric Reduction of Acetophenone Using Cp*IrCl[(S,S)-MsDPEN] Catalyst and Potassium Formate Solution as Hydrogen Source

3.36 g (40.0 mmol) of HCOOK as the hydrogen source, 1.044 mg (1.6 μmol) of Cp*IrCl[(S,S)-MsDPEN] as the catalyst, and 0.93 mL (8.0 mmol) of acetophenone were introduced in a 20 mL Schlenk tube, and the mixture was subjected to argon substitution. 2 mL of water was added and the resulting mixture was maintained at 50° C. for 24 hr while stirring. The organic phase was washed three times with 3 mL of water to give an optically-active alcohol. GC analysis of the reactant confirmed that 1-phenylethanol with optical purity of 93% ee was produced in 96% yield.

### Example A-2

### Asymmetric Reduction of Acetophenone Using Cp*IrCl[(S,S)-MsDPEN] Catalyst and Formic Acid-Triethylamine Mixture as Hydrogen Source

A formic acid-triethylamine mixture (molar ratio of HCOOH:Et₃N:substrate=3.1:2.6:1) as the hydrogen source, 10.44 mg (16.0 μmol) of Cp*IrCl[(S,S)-MsDPEN] as the catalyst, and 0.93 mL (8.0 mmol) of acetophenone were introduced in a 20 mL Schlenk tube, and the mixture was subjected to argon substitution and maintained at 50° C. for 24 hr while stirring. GC analysis of the reactant confirmed that 1-phenylethanol with optical purity of 75% ee was produced in 56% yield.

### Example A-4

### Asymmetric Reduction of Acetophenone Using Cp*Ir(OTf) [(S,S)-MsDPEN] Catalyst and Potassium Formate Solution as Hydrogen Source (Hydrogen Transfer Reaction Using Triflate Complex as Catalyst)

The reaction was performed under the same conditions as those in Example A-1, except that 1.227 mg (1.6 μmol) of Cp*Ir(OTf) [(S,S)-MsDPEN] was used as the catalyst. GC analysis of the reactant confirmed that 1-phenylethanol with optical purity of 93% ee was produced in 94% yield, demonstrating the effectiveness of using a triflate catalyst in combination with a potassium formate solution.

### Example A-6

### Asymmetric Reduction of Acetophenone Using Cp*IrCl[(R,R)-MsCYDN] Catalyst and Potassium Formate Solution as Hydrogen Source

The reaction was performed under the same conditions as those in Example A-1, except that 0.887 mg (1.6 μmol) of Cp*IrCl[(R,R)-MsCYDN] was used as the catalyst. GC analysis of the reactant confirmed that 1-phenylethanol with optical purity of 86% ee was produced in 90% yield.

### Example A-7

### Asymmetric Reduction of Acetophenone Using Cp*IrCl[(R,R)-MsCYDN] Catalyst and Formic Acid-Triethylamine Mixture as Hydrogen Source

The reaction was performed under the same conditions as those in Example A-2, except that 0.887 mg (1.6 μmol) of Cp*IrCl[(R,R)-MsCYDN] was used as the catalyst. GC analysis of the reactant showed that only a trace amount of 1-phenylethanol was detected.

### Example A-8

### Asymmetric Reduction of Acetophenone Using Cp*RhCl[(S,S)-MsDPEN] Catalyst and Potassium Formate Solution as Hydrogen Source (Use of Rhodium Complex)

The reaction was performed under the same conditions as those in Example A-1, except that 4.504 mg (8.0 μmol) of Cp*RhCl[(S,S)-MsDPEN] was used as the catalyst. GC analysis of the reactant confirmed that 1-phenylethanol with optical purity of 96% ee was produced in 83% yield.

### Comparative Example A-9

### Asymmetric Reduction of Acetophenone Using Cp*IrCl[(S,S)-TsDPEN] Catalyst and Potassium Formate Solution as Hydrogen Source (Comparison of Sulfonyl Substituent on Diamine Ligand)

The reaction was performed under the same conditions as those in Example A-1, except that 1.165 mg (1.6 μmol) of Cp*IrCl[(S,S)-TsDPEN] was used as the catalyst. GC analysis of the reactant confirmed that 1-phenylethanol with optical purity of 89% ee was produced in 27% yield. Comparison with Example A-1 demonstrated that it is superior to have a methyl group as the sulfonyl substituent on the diamine ligand.

### Comparative Example A-10

### Asymmetric Reduction of Acetophenone Using Cp*IrCl[(S,S)-TsDPEN] Catalyst and Formic Acid-Triethylamine Mixture as Hydrogen Source

The reaction was performed under the same conditions as those in Example A-2, except that 11.65 mg (16.0 μmol) of Cp*IrCl[(S,S)-TsDPEN] was used as the catalyst. GC analysis of the reactant showed that 1-phenylethanol with optical purity of 60% ee was produced in 33% yield. Comparison with Example A-2 demonstrated that it is superior to have a methyl group as the substituent on the sulfonyl group.

### Example B-1

### Asymmetric Reduction of α-hydroxyacetophenone Using Cp*IrCl[(S,S)-MsDPEN] Catalyst and Potassium Formate Solution as Hydrogen Source

3.36 g (40.0 mmol) of HCOOK as the hydrogen source, 1.044 mg (1.6 μmol) of Cp*IrCl[(S,S)-MsDPEN] as the catalyst, and 1.089 g (8.0 mmol) of unpurified α-hydroxyacetophenone were introduced in a 20 mL Schlenk tube, and the mixture was subjected to argon substitution. 2 mL of water and 2 mL of toluene were added and the resulting mixture was maintained at 50° C. for 24 hr while stirring. The organic phase was washed three times with 3 mL of water, and the toluene was distilled off under reduced pressure to give an optically-active alcohol. HPLC analysis of the reactant confirmed that 1-phenyl-1,2-ethanediol with optical purity of 94% ee was produced in 100% yield.

### Example B-2

### Asymmetric Reduction of α-hydroxyacetophenone Using Cp*IrCl[(S,S)-MsDPEN] Catalyst and Formic Acid-Triethylamine Mixture as Hydrogen Source

A formic acid-triethylamine mixture (molar ratio of HCOOH:Et₃N:substrate=3.1:2.6:1) as the hydrogen source, 1.044 mg (1.6 μmol) of Cp*IrCl[(S,S)-MsDPEN] as the catalyst, and 1.089 g (8.0 mmol) of α-hydroxyphenone were introduced in a 20 mL Schlenk tube, and the mixture was subjected to argon substitution and maintained at 50° C. for 24 hr while stirring. HPLC analysis of the reactant confirmed that 1-phenyl-1,2-ethanediol with optical purity of 66% ee was produced in 12% yield.

### Example B-3

### Asymmetric Reduction of α-hydroxyacetophenone Using Cp*Ir[(S,S)-MsDPEN] Catalyst and Potassium Formate Solution as Hydrogen Source (Use of Amide Complex)

The reaction was performed under the same conditions as those in Example B-1, except that 0.986 mg (1.6 μmol) of Cp*Ir[(S,S)-MsDPEN] was used as the catalyst. HPLC analysis of the reactant confirmed that 1-phenyl-1,2-ethanediol with optical purity of 94% ee was produced in 100% yield.

### Example B-4

### Asymmetric Reduction of α-hydroxyacetophenone Using Cp*Ir(OTf)[(S,S)-MsDPEN] Catalyst and Potassium Formate Solution as Hydrogen Source (Asymmetric Reduction Using Triflate Complex as Catalyst)

The reaction was performed under the same conditions as those in Example B-1, except that 1.227 mg (1.6 mmol) of Cp*Ir(OTf)[(S,S)-MsDPEN] was used as the catalyst. HPLC analysis of the reactant confirmed that 1-phenyl-1,2-ethanediol with optical purity of 90% ee was produced in 97% yield, demonstrating the effectiveness of using a triflate catalyst in combination with a potassium formate solution.

### Comparative Example B-5-1

### Asymmetric Hydrogenation of Purified β-hydroxyacetophenone Using Cp*Ir(OTf)[(S,S)-MsDPEN] Catalyst and Hydrogen Gas (Comparison Between Asymmetric Hydrogenation and Asymmetric Reduction)

1.532 mg (2.0 mmol) of Cp*Ir(OTf) [(S,S)-MsDPEN] and 1.361 g (10.0 mmol) of β-hydroxyacetophenone which was distilled and purified after the removal of trace amounts of acidic components by the treatment with a NaHCO₃ solution were introduced in an autoclave, and the mixture was subjected to argon substitution. 3.3 mL of methanol was introduced and deaeration was performed, then hydrogen gas was introduced at 10 atm and the resulting mixture was maintained at 60° C. for 24 hr while stirring. The solvent was distilled off under reduced pressure to give a crude product. HPLC analysis of the reactant confirmed that 1-phenyl-1,2-ethanediol with optical purity of 96% ee was produced in 100% yield.

### Comparative Example B-5-2

### Asymmetric Hydrogenation of Unpurified α-hydroxyacetophenone Using Cp*Ir(OTf)[(S,S)-MsDPEN] Catalyst and Hydrogen Gas (Comparison of Asymmetric-Hydrogenation Reaction Between Different Grades of Purification in Substrates)

The reaction was performed under the same conditions as those in Comparative Example B-5-1, except that as the ketone substrate, the reagent was used unpurified. HPLC analysis of the reactant confirmed that the yield of 1-phenyl-1,2-ethanediol was only 5%. It was demonstrated that in the title catalyst system, the purity of ketone substrates affects the reproducibility of the asymmetric hydrogenation.

### Example B-8

### Asymmetric Reduction of α-hydroxyacetophenone Using Cp*RhCl[(S,S)-MsDPEN] Catalyst and Formic Acid-Triethylamine Mixture as Hydrogen Source (Use of Rhodium Complex)

The reaction was performed under the same conditions as those in Example B-1, except that 2.252 mg (4.0 μmol) of Cp*RhCl[(S,S)-MsDPEN] was used as the catalyst. HPLC analysis of the reactant confirmed that 1-phenyl-1,2-ethanediol with optical purity of 98% ee was produced in 100% yield, demonstrating the superiority of using a rhodium complex in combination with a potassium formate solution.

### Comparative Example B-9

### Asymmetric Reduction of α-hydroxyacetophenone Using Cp*IrCl[(R,R)-TsDPEN] Catalyst and Potassium Formate Solution as Hydrogen Source (Comparison of Sulfonyl Substituent on Diamine Ligand)

The reaction was performed under the same conditions as those in Example B-1, except that 1.165 mg (1.6 μmol) of Cp*IrCl[(R,R)-TsDPEN] was used as the catalyst. HPLC analysis of the reactant confirmed that 1-phenyl-1,2-ethanediol with optical purity of 28% ee was produced in 30% yield. Comparison with Example B-1 demonstrated that it is superior to have a methyl group as the substituent on the sulfonyl group.

### Comparative Example B-11

### Asymmetric Reduction of α-hydroxyacetophenone Using Cp*IrCl[(S,S)-TsCYDN] Catalyst and Potassium Formate Solution as Hydrogen Source (Comparison of Diamine Ligand)

The reaction was performed under the same conditions as those in Example B-1, except that 1.008 mg (1.6 μmol) of Cp*IrCl[(S,S)-TsCYDN] was used as the catalyst. HPLC analysis of the reactant confirmed that 1-phenyl-1,2-ethanediol with optical purity of 68% ee was produced in 10% yield. Comparison with Example B-1 demonstrated the superiority of MsDPEN as the diamine ligand.

### Comparative Example B-14

### Asymmetric Reduction of α-hydroxyacetophenone Using RuCl[(R,R)-MsDPEN] (p-cymene) Catalyst and Potassium Formate Solution as Hydrogen Source (Use of Ruthenium Catalyst)

The reaction was performed under the same conditions as those in Example B-1, except that 0.896 mg (1.6 μmol) of RuCl[(R,R)-MsDPEN] (p-cymene) was used as the catalyst. HPLC analysis of the reactant confirmed that the yield of 1-phenyl-1,2-ethanediol was less than 1%. Comparison with Example B-1 demonstrated that the activity of the ruthenium complex is very low, so that the iridium complex having a methanesulfonyl diamine ligand is superior.

### Comparative Example B-15

### Asymmetric Reduction of α-hydroxyacetophenone Using Cp*IrCl[(R,R)— (R)-CsDPEN] Catalyst and Potassium Formate Solution As Hydrogen Source

The reaction was performed under the same conditions as those in Example B-1, except that 1.467 mg (1.6 μmol) of Cp*IrCl[(R,R)—(R)-CsDPEN] was used as the catalyst. HPLC analysis of the reactant confirmed that 1-phenyl-1,2-ethanediol with optical purity of 87% ee was produced in 40% yield, showing that the catalytic efficiency of the iridium complex having camphorsulfonyl DPEN as the ligand is insufficient for the asymmetric reduction of ketones having a functional group.

### Example C-1

### Asymmetric Reduction of β-hydroxypropiophenone Using Cp*IrCl[(S,S)-MsDPEN] Catalyst and Potassium Formate Solution as Hydrogen Source

3.36 g (40.0 mmol) of HCOOK as the hydrogen source, 2.609 mg (4.0 μmol) of Cp*IrCl[(S,S)-MsDPEN] as the catalyst, and 1.201 g (8.0 mmol) of β-hydroxypropiophenone were introduced in a 20 mL Schlenk tube, and the mixture was subjected to argon substitution. 2 mL of water was added and the resulting mixture was maintained at 50° C. for 24 hr while stirring. The organic phase was washed three times with 3 mL of water to give an optically-active alcohol. HPLC analysis of the reactant confirmed that 1-phenyl-1,3-propanediol with optical purity of 93% ee was produced in 99% yield.

### Comparative Example C-5

### Asymmetric Hydrogenation of β-hydroxypropiophenone Using Cp*Ir(OTf) [(S,S)-MsDPEN] Catalyst and Hydrogen Gas (Comparison Between Asymmetric Hydrogenation and Asymmetric Reduction)

6.127 mg (8.0 μmol) of Cp*Ir(OTf)[(S,S)-MsDPEN] and 1.201 g (8.0 mmol) of β-hydroxypropiophenone were introduced in an autoclave, and the mixture was subjected to argon substitution. 3.3 mL of methanol was introduced and deaeration was performed, then hydrogen gas was introduced at 10 atm and the resulting mixture was maintained at 60° C. for 24 hr while stirring. The solvent was distilled off under reduced pressure to give a crude product. HPLC analysis of the reactant confirmed that 1-phenyl-1,3-propanediol with optical purity of 75% ee was produced in 18% yield. Comparison with Example C-1 demonstrated the superiority of the asymmetric reduction using a potassium formate solution as the hydrogen source.

### Example C-6

### Asymmetric Reduction of β-hydroxypropiophenone Using Cp*Ir[(R,R)-MsCYDN] Catalyst and Potassium Formate Solution as Hydrogen Source (Use of MsCYDN Ligand)

The reaction was performed under the same conditions as those in Example C-1, except that 2.217 mg (4.0 μmol) of Cp*Ir[(R,R)-MsCYDN] was used as the catalyst. HPLC analysis of the reactant confirmed that 1-phenyl-1,3-propanediol with optical purity of 82% ee was produced in 95% yield.

### Comparative Example C-14

### Asymmetric Reduction of β-hydroxypropiophenone Using RuCl[(R,R)-MsDPEN] (p-cymene) Catalyst and Potassium Formate Solution as Hydrogen Source (Comparison Between Iridium Complex and Ruthenium Complex)

The reaction was performed under the same conditions as those in Example C-1, except that 2.240 mg (4.0 mmol) of RuCl[(R,R)-MsDPEN] (p-cymene) was used as the catalyst. HPLC analysis of the reactant confirmed that 1-phenyl-1,3-propanediol was produced in 4% yield. Comparison with Example C-1 demonstrated that the activity of the ruthenium complex is very low, so that the iridium complex having a methanesulfonyl diamine ligand is superior.

### Example D-1

### Asymmetric Reduction of α-chloroacetophenone Using Cp*IrCl[(S,S)-MsDPEN] Catalyst and Potassium Formate Solution as Hydrogen Source

3.36 g (40.0 mmol) of HCOOK as the hydrogen source, 1.044 mg (1.6 μmol) of Cp*IrCl[(S,S)-MsDPEN] as the catalyst, and 1.237 g (8.0 mmol) of α-chloroacetophenone were introduced in a 20 mL Schlenk tube, and the mixture was subjected to argon substitution. 2 mL of water and 2 ml of toluene were added and the resulting mixture was maintained at 50° C. for 24 hr while stirring. The organic phase was washed three times with 3 mL of water, and the toluene was distilled off under reduced pressure to give an optically-active alcohol. GC analysis of the reactant confirmed that 2-chloro-1-phenylethane-1-ol with optical purity of 92% ee was produced in 87% yield.

### Example D-2

### Asymmetric Reduction of α-chloroacetophenone Using Cp*IrCl[(S,S)-MsDPEN] Catalyst and Formic Acid-Triethylamine Mixture as Hydrogen Source

A formic acid-triethylamine mixture (molar ratio of HCOOH:Et₃N:substrate=3.1:2.6:1) as the hydrogen source, 1.044 mg (1.6 μmol) of Cp*IrCl[(S,S)-MsDPEN] as the catalyst, and 1.237 g (8.0 mmol) of α-chloroacetophenone were introduced in a 20 mL Schlenk tube, and the mixture was subjected to argon substitution, then maintained at 50° C. for 24 hr while stirring. NMR analysis of the reactant showed the disappearance of the raw materials, but signals derived from 2-chloro-1-phenylethane-1-ol of interest could not be confirmed and complex signals derived from the mixture were observed.

### Comparative Example D-5

### Asymmetric Hydrogenation of α-chloroacetophenone Using Cp*Ir(OTf)[(S,S)-MsDPEN] Catalyst and Hydrogen Gas (Comparison between asymmetric hydrogenation and asymmetric reduction)

3.064 mg (4.0 mmol) of Cp*Ir(OTf) [(S,S)-MsDPEN] and 1.237 g (8.0 mmol) of α-chloroacetophenone were introduced in an autoclave, and the mixture was subjected to argon substitution. 3.3 mL of methanol was introduced and deaeration was performed, then hydrogen gas was introduced at 10 atm and the resulting mixture was maintained at 60° C. for 24 hr while stirring. The solvent was distilled off under reduced pressure to give a crude product. GC analysis of the reactant confirmed that 2-chloro-1-phenylethane-1-ol with optical purity of 92% ee was produced in 39% yield. Comparison with Example D-1 demonstrated the superiority of the asymmetric reduction using a potassium formate solution as the hydrogen source.

### Example D-6

### Asymmetric Reduction of α-chloroacetophenone Using Cp*IrCl[(R,R)-MsCYDN] Catalyst and Potassium Formate Solution as Hydrogen Source (Utilization of MsCYDN Ligand)

The reaction was performed under the same conditions as those in Example D-1, except that 0.887 mg (1.6 μmol) of Cp*IrCl[(R,R)-MsCYDN] was used as the catalyst. GC analysis of the reactant confirmed that 2-chloro-1-phenylethane-1-ol with optical purity of 82% ee was produced in 92% yield.

### Example D-7

### Asymmetric Reduction of α-chloroacetophenone Using Cp*IrCl[(R,R)-MsCYDN] Catalyst and Formic Acid-Triethylamine Mixture as Hydrogen Source

The reaction was performed under the same conditions as those in Example D-2, except that 0.887 mg (1.6 μmol) of Cp*IrCl (R,R)-MsCYDN] was used as the catalyst. NMR analysis of the reactant showed the disappearance of the raw materials and the generation of a compound of unknown structure; 2-chloro-1-phenylethane-1-ol of interest could not be detected.

### Example D-8

### Asymmetric Reduction of α-chloroacetophenone Using Cp*RhCl[(S,S)-MsDPEN] Catalyst and Potassium Formate Solution as Hydrogen Source (Use of Rhodium Complex)

The reaction was performed under the same conditions as those in Example D-1, except that 4.504 mg (8.0 μmol) of Cp*RhCl[(S,S)-MsDPEN] was used as the catalyst. GC analysis of the reactant confirmed that 2-chloro-1-phenylethane-1-ol with optical purity of 97% ee was produced in 100% yield.

### Comparative Example D-9

### Asymmetric Reduction of α-Chloroacetophenone Using Cp*IrCl[(S,S)-TsDPEN] Catalyst and Potassium Formate Solution as Hydrogen Source (Comparison of Sulfonyl Substituent on Diamine Ligand)

The reaction was performed under the same conditions as those in Example D-1, except that 1.165 mg (1.6 μmol) of Cp*IrCl[(S,S)-TsDPEN] was used as the catalyst. GC analysis of the reactant confirmed that 2-chloro-1-phenylethane-1-ol with optical purity of 91% ee was produced in 26% yield. Comparison with Example D-1 demonstrated that it is superior to have a methyl group as the substituent on the sulfonyl group.

### Comparative Example D-11

### Asymmetric Reduction of α-chloroacetophenone Using Cp*IrCl[(R,R)-TsCYDN] Catalyst and Potassium Formate Solution as Hydrogen Source (Comparison of Diamine Ligand)

The reaction was performed under the same conditions as those in Example D-1, except that 1.008 mg (1.6 μmol) of Cp*IrCl[(R,R)-TsCYDN] was used as the catalyst. GC analysis of the reactant confirmed that 2-chloro-1-phenylethane-1-ol with optical purity of 80% ee was produced in 29% yield. Comparison with Example D-6 demonstrated the superiority of MsCYDN as the diamine ligand.

### Comparative Example D-12

### Asymmetric Reduction of α-chloroacetophenone Using RuCl[(R,R)-TsDPEN] (p-cymene) Catalyst and Potassium Formate Solution as Hydrogen Source (Comparison of Sulfonyl Substituent on Diamine Ligand)

The reaction was performed under the same conditions as those in Example D-1, except that 5.090 mg (8.0 μmol) of RuCl[(R,R)-TsDPEN] (p-cymene) was used as the catalyst. GC analysis of the reactant confirmed that 2-chloro-1-phenylethane-1-ol with optical purity of 86% ee was produced in 85% yield. Comparison with Example D-1 demonstrated that the activity of the ruthenium complex is very low, so that the iridium complex having a methanesulfonyl diamine ligand is superior.

### Example E-1

### Asymmetric Reduction of β-Chloropropiophenone Using Cp*IrCl[(S,S)-MsDPEN] Catalyst and Potassium Formate Solution as Hydrogen Source

3.36 g (40.0 mmol) of HCOOK as the hydrogen source, 2.609 mg (4.0 μmol) of Cp*IrCl[(S,S)-MsDPEN] as the catalyst, and 1.349 g (8.0 mmol) of β-chloropropiophenone were introduced in a 20 mL Schlenk tube, and the mixture was subjected to argon substitution. 2 mL of water and 2 ml of toluene were added and the resulting mixture was maintained at 50° C. for 24 hr while stirring. The organic phase was washed three times with 3 mL of water, and the toluene was distilled off under reduced pressure to give an optically-active alcohol. GC analysis of the reactant confirmed that 3-chloro-1-phenylpropane-1-ol with optical purity of 85% ee was produced in 94% yield.

### Comparative Example E-5

### Asymmetric Hydrogenation of β-chloropropiophenone Using Cp*Ir(OTf) [(S,S)-MsDPEN] Catalyst and Hydrogen Gas (Comparison Between Asymmetric Hydrogenation and Asymmetric Reduction)

6.127 mg (8.0 μmol) of Cp*Ir (OTf) [(S,S)-MsDPEN] and 1.249 g (8.0 mmol) of β-chloropropiophenone were introduced in an autoclave, and the mixture was subjected to argon substitution. 3.3 mL of methanol was introduced and deaeration was performed, then hydrogen gas was introduced at 10 atm and the resulting mixture was maintained at 60° C. for 24 hr while stirring. The solvent was distilled off under reduced pressure to give a crude product. GC analysis of the reactant confirmed that 3-chloro-1-phenylpropane-1-ol with optical purity of 77% ee was produced in 12% yield. Comparison with Example E-1 demonstrated the superiority of the asymmetric reduction using a potassium formate solution as the hydrogen source.

### Comparative Example E-13

### Asymmetric Hydrogenation of β-chloropropiophenone Using RuCl[(R,R)-TsDPEN] (p-cymene) Catalyst and Hydrogen Gas (Comparison Between Asymmetric Hydrogenation and Asymmetric Reduction)

5.010 mg (8.0 μmol) of RuCl[(R,R)-TsDPEN] (p-cymene) and 1.249 g (8.0 mmol) of β-chloropropiophenone were introduced in an autoclave, and the mixture was subjected to argon substitution. 3.3 mL of methanol was introduced and deaeration was performed, then hydrogen gas was introduced at 10 atm and the resulting mixture was maintained at 60° C. for 24 hr while stirring. The solvent was distilled off under reduced pressure to give a crude product. GC analysis of the reactant confirmed that 3-chloro-1-phenylpropane-1-ol with optical purity of 90% ee was produced in 9% yield. Comparison with Example E-1 demonstrated the superiority of the asymmetric reduction using a potassium formate solution as the hydrogen source.

### Example F-1-1

### Asymmetric Reduction of 4-Chromanone Using Cp*IrCl[(S,S)-MsDPEN] Catalyst and Potassium Formate Solution as Hydrogen Source

3.36 g (40.0 mmol) of HCOOK as the hydrogen source, 1.044 mg (1.6 μmol) of Cp*IrCl[(S,S)-MsDPEN] as the catalyst, and 1.185 g (8.0 mmol) of 4-chromanone were introduced in a 20 mL Schlenk tube, and the mixture was subjected to argon substitution. 2 mL of water and 2 ml of toluene were added and the resulting mixture was maintained at 50° C. for 24 hr while stirring. The organic phase was washed three times with 3 mL of water, and the toluene was distilled off under reduced pressure to give an optically-active alcohol. HPLC analysis of the reactant confirmed that 4-chromanol with optical purity of 95% ee was produced in 89% yield.

### Example F-1-2

### Asymmetric Reduction of 4-Chromanone Using Cp*IrCl[(S,S)-MsDPEN] Catalyst with Addition of Phase-Transfer Catalyst and Potassium Formate Solution as Hydrogen Source

The reaction was performed under the same conditions as those in Example F-1-1, except that 32 mg (100 μmol) of tetrabutylammonium bromide as the phase-transfer catalyst was added. HPLC analysis of the reactant confirmed that 4-chromanol with optical purity of 97% ee was produced in 99% yield, demonstrating that the activity and enantioselectivity of the main catalyst is improved by the addition of a phase-transfer catalyst.

### Example F-1-3

### Asymmetric Reduction of 4-Chromanone Using Cp*IrCl[(S,S)-MsDPEN] Catalyst with Addition of Phase-Transfer Catalyst and Potassium Formate Solution as Hydrogen Source (Reaction at S/C=10,000)

2.02 g (24.0 mmol) of HCOOK as the hydrogen source, 1.305 mg (2.0 μmol) of Cp*IrCl[(S,S)-MsDPEN] as the catalyst, 64.5 mg (0.20 mmol) of tetrabutylammonium bromide as the phase-transfer catalyst, and 2.96 g (20.0 mmol) of 4-chromanone were introduced in a 20 mL Schlenk tube, and the mixture was subjected to argon substitution. 4 mL of water and 2 ml of toluene were added and the resulting mixture was maintained at 50° C. for 24 hr while stirring. The organic phase was washed three times with 5 mL of water, and the toluene was distilled off under reduced pressure to give an optically-active alcohol. HPLC analysis of the reactant confirmed that 4-chromanol with optical purity of 98% ee was produced in 96% yield, demonstrating that the use of a potassium formate solution as the hydrogen source and the catalyst system in which Cp*IrCl[(S,S)-MsDPEN] catalyst is combined with tetrabutylammonium bromide exhibits high efficiency.

### Example F-2-1

### Asymmetric Reduction of 4-Chromanone Using Cp*IrCl[(S,S)-MsDPEN] Catalyst and Formic Acid-Triethylamine Mixture as Hydrogen Source

A formic acid-triethylamine mixture (molar ratio of HCOOH:Et₃N:substrate=3.1:2.6:1) as the hydrogen source, 1.044 mg (1.6 μmol) of Cp*IrCl[(S,S)-MsDPEN] as the catalyst, and 1.185 g (8.0 mmol) of 4-chromanone were introduced in a 20 mL Schlenk tube, and the mixture was subjected to argon substitution, then maintained at 50° C. for 24 hr while stirring. HPLC analysis of the reactant confirmed that 4-chromanol with optical purity of 88% ee was produced in 10% yield.

### Example F-2-2

### Asymmetric Reduction of 4-Chromanone Using Cp*IrCl[(S,S)-MsDPEN] Catalyst and Formic Acid-Triethylamine Mixture as Hydrogen Source

The reaction was performed under the same conditions as those in Example F-2-1, except that the amount of the catalyst was 2.610 mg (4.0 μmol). HPLC analysis of the reactant confirmed that 4-chromanol with optical purity of 95% ee was produced in 89% yield.

### Example F-6

### Asymmetric Reduction of 4-Chromanone Using Cp*IrCl[(R,R)-MsCYDN] Catalyst and Potassium Formate Solution as Hydrogen Source (Utilization of MsCYDN Ligand)

The reaction was performed under the same conditions as those in Example F-1-1, except that 0.887 mg (1.6 μmol) of Cp*IrCl[(R,R)-MsCYDN] was used as the catalyst. HPLC analysis of the reactant confirmed that 4-chromanol with optical purity of 94% ee was produced in 44%, yield, demonstrating that the combination of the Cp*IrCl[(R,R)-MsCYDN] complex with a potassium formate solution exhibits a moderate level of catalytic activity.

### Comparative Example F-9

### Asymmetric Reduction of 4-chromanone Using Cp*IrCl[(S,S)-TsDPEN] Catalyst and Potassium Formate Solution as Hydrogen Source (Comparison of Sulfonyl Substituent on Diamine Ligand)

The reaction was performed under the same conditions as those in Example F-1-1, except that 1.165 mg (1.6 μmol) of Cp*IrCl[(S,S)-TsDPEN] was used as the catalyst. HPLC analysis of the reactant confirmed that 4-chromanol with optical purity of 94% ee was produced in 19% yield. Comparison with Example F-1-1 demonstrated that it is superior to have a methyl group as the substituent on the sulfonyl group.

### Comparative Example F-11

### Asymmetric Reduction of 4-chromanone Using Cp*IrCl[(R,R)-TsCYDN] Catalyst and Potassium Formate Solution as Hydrogen Source (Comparison of Diamine Ligand)

The reaction was performed under the same conditions as those in Example F-1-1, except that 1.008 mg (1.6 mmol) of Cp*IrCl[(R,R)-TsCYDN] was used as the catalyst. HPLC analysis of the reactant confirmed that 4-chromanol with optical purity of 71% ee was produced in 6% yield. Comparison with Example F-1-1 demonstrated the superiority of MsDPEN as the diamine ligand.

### Comparative Example F-14

### Asymmetric Reduction of 4-chromanone Using RuCl[(R,R)-MsDPEN] (p-cymene) Catalyst and Potassium Formate Solution as Hydrogen Source (Comparison Between Ruthenium Catalyst and Iridium Catalyst)

The reaction was performed under the same conditions as those in Example F-1-1, except that 0.896 mg (1.6 μmol) of RuCl[(R,R)-MsDPEN] (p-cymene) was used as the catalyst. HPLC analysis of the reactant confirmed that 4-chromanol with optical purity of 75% ee was produced in 9% yield. It was demonstrated that the ruthenium complex has very low activity in the asymmetric reduction of ketone substrates, so that the iridium complex having a methanesulfonyl diamine ligand is superior.

### Example G-1

### Asymmetric Reduction of Ethyl Benzoylacetate Using Cp*IrCl[(S,S)-MsDPEN] Catalyst and Potassium Formate Solution as Hydrogen Source

3.36 g (40.0 mmol) of HCOOK as the hydrogen source, 1.044 mg (1.6 μmol) of Cp*IrCl[(S,S)-MsDPEN] as the catalyst, and 1.586 g (8.0 mmol) of ethyl benzoylacetate were introduced in a 20 mL Schlenk tube, and the mixture was subjected to argon substitution. 2 mL of water was added and the resulting mixture was maintained at 50° C. for 24 hr while stirring. The organic phase was washed three times with 3 mL of water to give an optically-active alcohol. HPLC analysis of the reactant confirmed that ethyl 3-phenyl-3-hydroxypropionate with optical purity of 93% ee was produced in 98% yield.

### Example G-2

### Asymmetric Reduction of Ethyl Benzoylacetate Using Cp*IrCl[(S,S)-MsDPEN] Catalyst and Formic Acid-Triethylamine Mixture as Hydrogen Source

A formic acid-triethylamine mixture (molar ratio of HCOOH:Et₃N:substrate=3.1:2.6:1) as the hydrogen source, 5.128 mg (8.0 μmol) of Cp*IrCl[(S,S)-MsDPEN] as the catalyst, and 1.586 g (8.0 mmol) of ethyl benzoylacetate were introduced in a 20 mL Schlenk tube, and the mixture was subjected to argon substitution, then maintained at 50° C. for 24 hr while stirring. HPLC analysis of the reactant confirmed that ethyl 3-phenyl-3-hydroxypropionate with optical purity of 78% ee was produced in 51% yield.

### Comparative Example G-5

### Asymmetric Hydrogenation of Ethyl Benzoylacetate Using Cp*Ir (OTf) [(S,S)-MsDPEN] Catalyst and Hydrogen Gas (Comparison Between Asymmetric Hydrogenation and Asymmetric Reduction)

6.127 mg (8.0 μmol) of Cp*Ir (OTf) [(S,S)-MsDPEN] and 1.586 g (8.0 mmol) of ethyl benzoylacetate were introduced in an autoclave, and the mixture was subjected to argon substitution. 3.3 mL of methanol was introduced and deaeration was performed, then hydrogen gas was introduced at 10 atm and the resulting mixture was maintained at 60° C. for 24 hr while stirring. The solvent was distilled off under reduced pressure to give a crude product. GC analysis of the reactant confirmed that ethyl 3-phenyl-3-hydroxypropionate with optical purity of 95% ee was produced in 81% yield. Comparison with Example G-1 demonstrated that the catalytic activity of this comparative Example was approximately ⅕ of that in the asymmetric reduction using a potassium formate solution as the hydrogen source shown in Example G-1.

### Comparative Example G-10

### Asymmetric Reduction of Ethyl Benzoylacetate Using Cp*IrCl[(S,S)-TsDPEN] Catalyst and Formic Acid-Triethylamine Mixture as Hydrogen Source (Comparison of Sulfonyl Substituent on Diamine Ligand)

The reaction was performed under the same conditions as those in Example G-2, except that 5.825 mg (8.0 μmol) of Cp*IrCl[(S,S)-TsDPEN] was used as the catalyst. HPLC analysis of the reactant confirmed that ethyl 3-phenyl-3-hydroxypropionate with optical purity of 69% ee was produced in 50% yield. Comparison with Example G-2 demonstrated the superiority of MsDPEN as the diamine ligand.

### Comparative Example G-14

### Asymmetric Reduction of Ethyl Benzoylacetate Using RuCl[(R,R)-MsDPEN] (p-cymene) Catalyst and Potassium Formate Solution as Hydrogen Source (Comparison Between Ruthenium Catalyst and Iridium Catalyst)

The reaction was performed under the same conditions as those in Example G-1, except that 0.896 mg (1.6 μmol) of RuCl[(R,R)-MsDPEN] (p-cymene) was used as the catalyst. HPLC analysis of the reactant confirmed that ethyl 3-phenyl-3-hydroxypropionate with optical purity of 91% ee was produced in 18% yield. Comparison with Example G-1 demonstrated that the activity of the ruthenium complex was low, so that the iridium complex with a methanesulfonyl diamine ligand was superior.

### Example H-1

### Asymmetric Reduction of Ethyl 3-oxo-3-(2-fluorophenyl)propionate Using Cp*IrCl[(S,S)-MsDPEN] Catalyst and Potassium Formate Solution as Hydrogen Source

3.36 g (40.0 mmol) of HCOOK as the hydrogen source, 1.044 mg (1.6 μmol) of Cp*IrCl[(S,S)-MsDPEN] as the catalyst, and 1.682 g (8.0 mmol) of ethyl 3-oxo-3-(2-fluorophenyl) propionate were introduced in a 20 mL Schlenk tube, and the mixture was subjected to argon substitution. 2 mL of water was added and the resulting mixture was maintained at 50° C. for 24 hr while stirring. The organic phase was washed three times with 3 mL of water to give an optically-active alcohol. HPLC analysis of the reactant confirmed that ethyl 3-(2-fluorophenyl)-3-hydroxypropionate with optical purity of 59% ee was produced in 100% yield.

### Comparative Example H-5

### Asymmetric Hydrogenation of Ethyl 3-oxo-3-(2-fluorophenyl)propionate Using Cp*Ir(OTf)[(S,S)-MsDPEN] Catalyst and Hydrogen Gas (Comparison Between Asymmetric Hydrogenation and Asymmetric Reduction)

6.127 mg (8.0 mmol) of Cp*Ir (OTf) [(S,S)-MsDPEN] and 1.682 g (8.0 mmol) of ethyl 3-oxo-3-(2-fluorophenyl)propionate were introduced in an autoclave, and the mixture was subjected to argon substitution. 3.3 mL of methanol was introduced and deaeration was performed, then hydrogen gas was introduced at 10 atm and the resulting mixture was maintained at 60° C. for 24 hr while stirring. The solvent was distilled off under reduced pressure to give a crude product. GC analysis of the reactant confirmed that ethyl 3-(2-fluorophenyl)-3-hydroxypropionate with optical purity of 65% ee was produced in 40% yield. Comparison with Example H-1 demonstrated the superiority of the asymmetric reduction using a potassium formate solution as the hydrogen source.

### Example I-1

### Asymmetric Reduction of ethyl 3-oxo-3-(4-pyridyl)propionate Using Cp*IrCl[(S,S)-MsDPEN] Catalyst and Potassium Formate Solution as Hydrogen Source

3.36 g (40.0 mmol) of HCOOK as the hydrogen source, 1.044 mg (1.6 μmol) of Cp*IrCl[(S,S)-MsDPEN] as the catalyst, and 1.546 g (8.0 mmol) of ethyl 3-oxo-3-(4-pyridyl) propionate were introduced in a 20 mL Schlenk tube, and the mixture was subjected to argon substitution. 2 mL of water and 2 mL of toluene were added and the resulting mixture was maintained at 50° C. for 24 hr while stirring. The organic phase was washed three times with 3 mL of water, the solvent was distilled off under reduced pressure, to give an optically-active alcohol. HPLC analysis of the reactant confirmed that ethyl 3-hydroxy-3-(4-pyridyl) propionate with optical purity of 84% ee was produced in 100% yield.

### Example I-2

### Asymmetric Reduction of ethyl 3-oxo-3-(4-pyridyl)propionate Using Cp*IrCl[(S,S)-MsDPEN] Catalyst and Formic Acid-Triethylamine Mixture as Hydrogen Source

A formic acid-triethylamine mixture (molar ratio of HCOOH:Et₃N:substrate=3.1:2.6:1) as the hydrogen source, 5.128 mg (8.0 μmol) of Cp*IrCl[(S,S)-MsDPEN] as the catalyst, and 1.546 g (8.0 mmol) of ethyl 3-oxo-3-(4-pyridyl) propionate were introduced in a 20 mL Schlenk tube, and the mixture was subjected to argon substitution, then maintained at 50° C. for 24 hr while stirring. HPLC analysis of the reactant confirmed that ethyl 3-hydroxy-3-(4-pyridyl)propionate with optical purity of 80% ee was produced in 11% yield.

### Comparative Example I-11

### Asymmetric Reduction of ethyl 3-oxo-3-(4-pyridyl)propionate Using Cp*IrCl[(R,R)-TsCYDN] Catalyst and Potassium Formate Solution as Hydrogen Source (Comparison of Diamine Ligand)

The reaction was performed under the same conditions as those in Example I-1, except that 5.042 mg (8.0 μmol) of Cp*IrCl[(R,R)-TsCYDN] was used as the catalyst. HPLC analysis of the reactant confirmed that ethyl 3-hydroxy-3-(4-pyridyl)propionate with optical purity of 66% ee was produced in 10% yield. Comparison with Example I-1 demonstrated the superiority of MsDPEN as the diamine ligand.

### Comparative Example I-12

### Asymmetric Reduction of ethyl 3-oxo-3-(4-pyridyl)propionate Using RuCl[(R,R)-TsDPEN] (p-cymene) Catalyst and Potassium Formate Solution as Hydrogen Source (Comparison Between Ruthenium Complex And Iridium Complex)

The reaction was performed under the same conditions as those in Example I-1, except that 1.018 mg (1.6 μmol) of RuCl[(R,R)-TsDPEN] (p-cymene) was used as the catalyst. HPLC analysis of the reactant confirmed that only a trace amount of ethyl 3-hydroxy-3-(4-pyridyl) propionate was produced. Comparison with Example I-1 demonstrated that the activity of the ruthenium complex is very low in the asymmetric reduction of ketoester substrates, so that the iridium complex having a methane sulfonyl diamine ligand is superior.

### Comparative Example I-14

### Asymmetric Reduction of ethyl 3-oxo-3-(4-pyridyl)propionate Using RuCl[(R,R)-MsDPEN] (p-cymene) Catalyst and Potassium Formate Solution as Hydrogen Source (Use of Ruthenium Catalyst)

The reaction was performed under the same conditions as those in Example I-1, except that 4.481 mg (8.0 μmol) of RuCl[(R,R)-MsDPEN] (p-cymene) was used as the catalyst. HPLC analysis of, the reactant confirmed that ethyl 3-(4-pyridyl)-3-hydroxypropionate with optical purity of 75% ee was produced in 16% yield. Comparison with Example I-1 demonstrated that the activity of the ruthenium complex is very low, so that the iridium complex having a methanesulfonyl diamine ligand is superior.

### Example J-4

### Asymmetric Reduction of ethyl 3-oxo-3-(2-thienyl)propionate Using Cp*Ir(OTf) [(S,S)-MsDPEN] Catalyst and Potassium Formate Solution as Hydrogen Source (Asymmetric Reduction Using Triflate Complex as Catalyst)

3.36 g (40.0 mmol) of HCOOK as the hydrogen source, 1.227 mg (1.6 μmol) of Cp*Ir (OTf) [(S,S)-MsDPEN] as the catalyst, and 1.586 g (8.0 mmol) of ethyl 3-oxo-3-(2-thienyl) propionate were introduced in a 20 mL Schlenk tube, and the mixture was subjected to argon substitution. 2 mL of water was added and the resulting mixture was maintained at 50° C. for 24 hr while stirring. The organic phase was washed three times with 3 mL of water to give an optically-active alcohol. HPLC analysis of the reactant confirmed that ethyl 3-hydroxy-3-(2-thienyl)propionate with optical purity of 96% ee was produced in 90% yield.

### Comparative Example J-5

### Asymmetric Hydrogenation of ethyl 3-oxo-3-(2-thienyl)propionate Using Cp*Ir (OTf) [(S,S)-MsDPEN] Catalyst and Hydrogen Gas (Comparison Between Asymmetric Hydrogenation and Asymmetric Reduction)

6.127 mg (8.0 μmol) of Cp*Ir (OTf) [(S,S)-MsDPEN] and 1.586 g (8.0 mmol) of ethyl 3-oxo-3-(2-thienyl) propionate were introduced in an autoclave, and the mixture was subjected to argon substitution. 3.3 mL of methanol was introduced and deaeration was performed, then hydrogen gas was introduced at 10 atm and the resulting mixture was maintained at 60° C. for 24 hr while stirring. The solvent was distilled off under reduced pressure to give a crude product. GC analysis of the reactant confirmed that ethyl 3-hydroxy-3-(2-thienyl)propionate with optical purity of 97% ee was produced in 22% yield. Comparison with Example J-4 demonstrated that the catalytic activity of this comparative Example was only approximately 1/20 of that in the asymmetric reduction using a potassium formate solution as the hydrogen source shown in Example J-4.

### Example K-4

### Asymmetric Reduction of methyl 3-benzoylpropionate Using Cp*Ir(OTf)[(S,S)-MsDPEN] Catalyst and Potassium Formate Solution as Hydrogen Source (Asymmetric Reduction Using Triflate Complex as Catalyst)

3.36 g (40.0 mmol) of HCOOK as the hydrogen source, 3.068 mg (4.0 μmol) of Cp*Ir(OTf)[(S,S)-MsDPEN] as the catalyst, and 1. 538 g (8.0 mmol) of methyl 3-benzoylpropionate were introduced in a 20 mL Schlenk tube, and the mixture was subjected to argon substitution. 2 mL of water was added and the resulting mixture was maintained at 50° C. for 24 hr while stirring. The organic phase was washed three times with 3 mL of water to give a crude product.

NMR measurement showed that the crude product is a 1:1 mixture of methyl4-hydroxy-4-phenylbutanoic acid which is an optically-active alcohol and optically-active γ-phenyl-γ-butyrolactone which is generated by ring-closing of the former compound. The obtained mixture was treated with 0.152 g (0.80 mmol) of p-toluenesulfonic acid monohydrate in a diethylether solvent; HPLC measurement and NMR measurement of the resulting product confirmed that γ-phenyl-γ-butyrolactone with optical purity of 85% ee was produced in 96% yield.

### Comparative Example K-5

Asymmetric Hydrogenation of methyl 3-benzoylpropionate Using Cp*Ir (OTf) [(S,S)-MsDPEN] Catalyst and Hydrogen Gas (Comparison Between Asymmetric Hydrogenation and Asymmetric Reduction)

6.127 mg (8.0 μmol) of Cp*Ir (OTf) [(S,S)-MsDPEN] and 1.538 g (8.0 mmol) of methyl 3-benzoylpropionate were introduced in an autoclave, and the mixture was subjected to argon substitution. 3.3 mL of methanol was introduced and deaeration was performed, then hydrogen gas was introduced at 10 atm and the resulting mixture was maintained at 60° C. for 24 hr while stirring. The solvent was distilled off under reduced pressure to give a crude product. NMR measurement confirmed that the crude product is a mixture in a weight ratio of 1:1 of methyl 4-hydroxy-4-phenylbutanoic acid which is an optically-active alcohol and optically-active γ-phenyl-γ-butyrolactone which is generated by ring-closing of the former compound, produced in a yield of 3%. Comparison with Example K-4 demonstrated the superiority of the asymmetric reduction using a potassium formate solution as the hydrogen source.

### Example L-1

### Asymmetric Reduction of 1,1,1-trifluoroacetone Using Cp*IrCl[(S,S)-MsDPEN] Catalyst and Potassium Formate Solution as Hydrogen Source

3.36 g (40.0 mmol) of HCOOK as the hydrogen source, and 1.044 mg (1.6 μmol) of Cp*IrCl[(S,S)-MsDPEN] as the catalyst were introduced in a 20 mL Schlenk tube, and the mixture was subjected to argon substitution. 0.717 mg (8.0 mmol) of 1,1,1-trifluoroacetone and 2 mL of water were added and the resulting mixture was maintained at 30° C. for 24 hr while stirring. The reactant was distilled under normal pressure to give an optically-active alcohol in 73% yield. To measure the optical purity of the product, it was reacted with 1.50 mL (8.0 mmol) of (R)-(−)-α-methoxy-α-trifluoromethylphenylacetyl chloride in a pyridine solvent and stirred at room temperature overnight. The reactant solution was diluted with ethyl acetate and washed with water; GC analysis of the product confirmed that it has an optical purity of 87% ee.

### Example M-1

### Asymmetric Reduction of α-(benzoylamino)acetophenone Using Cp*IrCl[(S,S)-MsDPEN] Catalyst and Potassium Formate Solution as Hydrogen Source

3.36 g (40.0 mmol) of HCOOK as the hydrogen source, 2.609 mg (4.0 μmol) of Cp*IrCl[(S,S)-MsDPEN] as the catalyst, and 1.914 g (8.0 mmol) of α-(benzoylamino)acetophenone were introduced in a 20 mL Schlenk tube, and the mixture was subjected to argon substitution. 2 mL of water, 2 mL of toluene, and 2 mL of THF were added and the resulting mixture was maintained at 50° C. for 24 hr while stirring. The organic phase was washed three times with 3 mL of water, and the solvent was distilled off under reduced pressure to give an optically-active alcohol. HPLC analysis of the reactant confirmed that 2-(benzoylamino)-1-phenylethanol with optical purity of 91% ee was produced in 100% yield.

### Example M-2

### Asymmetric Reduction of α-(benzoylamino)acetophenone Using Cp*IrCl[(S,S)-MsDPEN] Catalyst and Formic Acid-Triethylamine Mixture as Hydrogen Source

A formic acid-triethylamine mixture (molar ratio of HCOOH:Et₃N:substrate=3.1:2.6:1) as the hydrogen source, 5.128 g (8.0 μmol) of Cp*IrCl[(S,S)-MsDPEN] as the catalyst, and 1.914 g (8.0 mmol) of α-(benzoylamino)acetophenone were introduced in a 20 mL Schlenk tube, and the mixture was subjected to argon substitution, then maintained at 30° C. for 24 hr while stirring. HPLC analysis of the reactant confirmed that 2-(benzoylamino)-1-phenylethanol was produced in 43% yield.

### Comparative Example M-5

### Asymmetric Hydrogenation of α-(benzoylamino)acetophenone Using Cp*Ir(OTf)[(S,S)-MsDPEN] Catalyst and Hydrogen Gas (Comparison Between Asymmetric Hydrogenation and Asymmetric Reduction)

6.127 mg (8.0 μmol) of Cp*Ir(OTf)[(S,S)-MsDPEN] and 1.914 g (8.0 mmol) of α-(benzoylamino)acetophenone were introduced in an autoclave, and the mixture was subjected to argon substitution. 3.3 mL of methanol was introduced and deaeration was performed, then hydrogen gas was introduced at 10 atm and the resulting mixture was maintained at 60° C. for 24 hr while stirring. The solvent was distilled off under reduced pressure to give a crude product. HPLC analysis of the reactant confirmed that 2-(benzoylamino)-1-phenylethanol with optical purity of 85% ee was produced in 55% yield, and the comparison with Example M-1 demonstrated the superiority of the asymmetric reduction using a potassium formate solution as the hydrogen source.

### Example N-1

### Asymmetric Reduction of α-(benzyloxycarbonylamino)acetophenone Using Cp*IrCl[(S,S)-MsDPEN] Catalyst and Potassium Formate Solution as Hydrogen Source

3.36 g (40.0 mmol) of HCOOK as the hydrogen source, 2.609 mg (4.0 μmol) of Cp*IrCl[(S,S)-MsDPEN] as the catalyst, and 2.154 g (8.0 mmol) of α-(benzyloxycarbonylamino)acetophenone were introduced in a 20 mL Schlenk tube, and the mixture was subjected to argon substitution. 2 mL of water and 2 mL of toluene were added and the resulting mixture was maintained at 50° C. for 24 hr while stirring. The organic phase was washed three times with 3 mL of water, and the solvent was distilled off under reduced pressure to give an optically-active alcohol. HPLC analysis of the reactant confirmed that 2-(benzyloxycarbonylamino)-1-phenylethanol with optical purity of 96% ee was produced in 1000 yield.

### Example N-2

### Asymmetric Reduction of α-(benzyloxycarbonylamino)acetophenone Using Cp*IrCl[(S,S)-MsDPEN] Catalyst and Formic Acid-Triethylamine Mixture as Hydrogen Source

A formic acid-triethylamine mixture (molar ratio of HCOOH:Et₃N:substrate=3.1:2.6:1) as the hydrogen source, 5.128 mg (8.0 μmol) of Cp*IrCl[(S,S)-MsDPEN] as the catalyst, and 2.154 g (8.0 mmol) of α-(benzyloxycarbonylamino) acetophenone were introduced in a 20 mL Schlenk tube, and the mixture was subjected to argon substitution, then maintained at 30° C. for 24 hr while stirring. HPLC analysis of the reactant confirmed that 2-(benzyloxycarbonylamino)-1-phenylethanol was produced in 9% yield.

### Comparative Example N-5

### Asymmetric Hydrogenation of α-(benzyloxycarbonylamino) acetophenone Using Cp*Ir (OTf) [(S,S)-MsDPEN] Catalyst and Hydrogen Gas (Comparison Between Asymmetric Hydrogenation and Asymmetric Reduction)

6.127 mg (8.0 μmol) of Cp*Ir (OTf) [(S,S)-MsDPEN] and 1.914 g (8.0 mmol) of α-(benzyloxycarbonylamino) acetophenone were introduced in an autoclave, and the mixture was subjected to argon substitution. 3.3 mL of methanol was introduced and deaeration was performed, then hydrogen gas was introduced at 10 atm and the resulting mixture was maintained at 60° C. for 24 hr while stirring. The solvent was distilled off under reduced pressure to give a crude product. HPLC analysis of the reactant confirmed that 2-(benzyloxycarbonylamino)-1-phenylethanol with optical purity of 87% ee was produced in 46% yield, and the comparison with Example N-1 demonstrated the superiority of the asymmetric reduction using a potassium formate solution as the hydrogen source.

### Example O-1

### Asymmetric Reduction of 2-hydroxy-1-(2-furyl)ethan-1-one Using Cp*IrCl[(S,S)-MsDPEN] Catalyst and Potassium Formate Solution as Hydrogen Source

3.36 g (40.0 mmol) of HCOOK as the hydrogen source, 5.218 mg (8.0 μmol) of Cp*IrCl[(S,S)-MsDPEN] as the catalyst, and 1.009 g (8.0 mmol) of 2-hydroxy-1-(2-furyl)ethan-1-one were introduced in a 20 mL Schlenk tube, and the mixture was subjected to argon substitution. 2 mL of water and 2 mL of toluene were added and the resulting mixture was maintained at 50° C. for 24 hr while stirring. The organic phase was washed three times with 3 mL of water, and the solvent was distilled off under reduced pressure to give an optically-active alcohol. HPLC analysis of the reactant confirmed that 1-(2-furyl)-1,2-ethanediol with optical purity of 94% ee was produced in 100% yield.

### Comparative Example O-5

### Asymmetric Hydrogenation of 2-hydroxy-1-(2-furyl)ethan-1-one Using Cp*Ir (OTf) [(S,S)-MsDPEN] Catalyst and Hydrogen Gas (Comparison Between Asymmetric Hydrogenation and Asymmetric Reduction)

6.127 mg (8.0 μmol) of Cp*Ir(OTf)[(S,S)-MsDPEN] and 1.009 g (8.0 mmol) of 2-hydroxy-1-(2-furyl)ethan-1-one were introduced in an autoclave, and the mixture was subjected to argon substitution. 3.3 mL of methanol was introduced and deaeration was performed, then hydrogen gas was introduced at 10 atm and the resulting mixture was maintained at 60° C. for 24 hr while stirring. The solvent was distilled off under reduced pressure to give a crude product. HPLC analysis of the reactant confirmed that 1-(2-furyl)-1,2-ethanediol with optical purity of 70% ee was produced in 12% yield, and the comparison with Example O-1 demonstrated the superiority of the asymmetric reduction using a potassium formate solution as the hydrogen source.

### Example P-1

### Asymmetric Reduction of 3-hydroxy-1-(2-thienyl)propanone Using Cp*IrCl[(S,S)-MsDPEN] Catalyst and Potassium Formate Solution as Hydrogen Source

3.36 g (40.0 mmol) of HCOOK as the hydrogen source, 2.609 mg (4.0 μmol) of Cp*IrCl[(S,S)-MsDPEN] as the catalyst, and 1.250 mg (8.0 mmol) of 3-hydroxy-1-(2-thienyl) propanone were introduced in a 20 mL Schlenk tube, and the mixture was subjected to argon substitution. 2 mL of water was added and the resulting mixture was maintained at 50° C. for 24 hr while stirring. The organic phase was washed three times with 3 mL of water, to give an optically-active alcohol. GC analysis of the reactant confirmed that 1-(2-thienyl)-1,3-propanediol with optical purity of 91% ee was produced in 72% yield.

The organic metal compound of the present invention can be utilized for the preparation of optically-active alcohols used as synthetic intermediates of various types of medical, agricultural or general-purpose chemicals.

1. (canceled)

2. (canceled)

3. (canceled)

4. A process for preparing optically active alcohols by asymmetric reduction of ketone substrates, wherein a ketone substrate is reacted with a hydrogen-donating compound under the presence of an organic metal compound represented by the general formula (2): 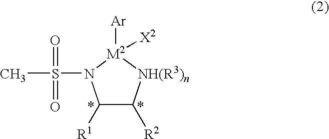
wherein R¹ and R² may be mutually identical or different, and are an alkyl group, a phenyl group, a naphthyl group, a cycloalkyl group, or an alicyclic ring formed by binding R¹ and R², which may have a substituent; R³ is a hydrogen atom or an alkyl group; Ar is a cyclopentadienyl group or a benzene ring group, which may have a substituent, bound to M² via a π bond; X² is a hydrido group or an anionic group; M² is rhodium or iridium; n is 0 or 1, and X² is absent when n is 0;
and * denotes asymmetric carbon.

5. The process according to claim 4, wherein R³ is a hydrogen atom and M² is iridium in the general formula (2).

6. The process according to claim 4, wherein a formate is used as a hydrogen-donating compound, and water or water/organic solvent is used as a solvent.

7. The process according to claim 4, wherein a phase-transfer catalyst is additionally added.

8. The process according to claim 4, wherein a ketone having a hydroxyl group at the α-position or the β-position of the ketone is asymmetrically reduced.

9. The process according to claim 4, wherein a ketone having a halogen at the α-position or the β-position of the ketone is asymmetrically reduced.

10. The process according to claim 4, wherein a ketone having a carbon-carbon multiple bond at the α-position or the β-position of the ketone is asymmetrically reduced.

11. The process according to claim 4, wherein a ketone having an ester group at the α-position or the β-position of the ketone, or a ketone having an ester group at the carbonyl carbon of the ketone is asymmetrically reduced.

12. The process according to claim 4, wherein a ketone having a carboxylic amide group at the α-position or the β-position of the ketone, or a ketone having a carboxylic amide group at the carbonyl carbon of the ketone is asymmetrically reduced.

13. The process according to claim 4, wherein a ketone having an amino group at the α-position or the β-position of the ketone is asymmetrically reduced.

14. The process according to claim 4, wherein 1,2-diketone or 1,3-diketone is asymmetrically reduced.

15. The process according to of claim 4, wherein a cyclic ketone is asymmetrically reduced.
